(19) United States
(12) Patent Application Publication
(10) Pub. No.: US 2025/0255903 A1
(43) Pub. Date: Aug. 14, 2025

(54) METHOD OF TREATING PARKINSON'S DISEASE WITH EXPANDED NATURAL KILLER CELLS

(71) Applicant: NKMAX CO., LTD., Gyeonggi-do (KR)

(72) Inventors: Paul Y. Song, Santa Monica, CA (US); Sang Woo Park, Gyeonggi-do (KR); Yong Man Kim, Gyeonggi-do (KR)

(21) Appl. No.: 18/992,936

(22) PCT Filed: Jul. 11, 2023
(86) PCT No.: PCT/US2023/070004

§ 371 Date: Jan. 9, 2025

Related U.S. Application Data
(60) Provisional application No. 63/501,954, filed on May 12, 2023.
(60) Provisional application No. 63/388,539, filed on Jul. 12, 2022.

(51) Int. Cl. A61K 35/17; A61P 25/16; C12N 5/0783

(52) U.S. Cl. CPC A61K 35/17; A61P 25/16; C12N 5/0646; C12N 2501/2302; C12N 2501/2321; C12N 2502/1107; C12N 2502/30

(57) ABSTRACT

Provided herein is a method for treating Parkinson's disease (PD). The method can include identifying a subject and treating the subject with expanded natural killer cells (NKs). Also provided is a composition for treating PD.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

The present disclosure relates to a method for treating Parkinson's disease with high-purity natural killer cells.

### BACKGROUND

Natural killer (NK) cells have proven to be promising candidates for use in adoptive cell therapy (ACT) due to their high cytotoxicity and lower risk than T-cells. One general approach to NK ACT has been the administration of autologous NK cells expanded ex vivo.

Parkinson's disease (PD) is a neurodegenerative disease affecting the motor system. Current treatments for PD are associated with alleviation of symptoms and managing the dopamine deficit.

### SUMMARY

This application is related to methods of producing high-purity natural killer cells, and cell therapeutic compositions for treating PD comprising high-purity natural killer cells and cytokines. Any features, structures, or steps disclosed herein can be replaced with or combined with any other features, structures, or steps disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

In some embodiments, a method of treating Parkinson's disease (PD) in a subject is provided. In some embodiments, the method comprises identifying a subject, wherein the subject has PD; and administering to the subject a therapeutically effective amount of an autologous natural killer cell (NK) cell population.

In some embodiments, a method of treating PD in a subject is provided. In some embodiments, the method comprises: identifying a subject, wherein the subject has PD; and administering to the subject an expanded NK cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a method of cell therapy is provided, comprising: identifying a subject, wherein the subject has PD; and administering to the subject an expanded NK cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a population of expanded NK cells is provided. In some embodiments, the NK cells were expanded by a method that comprises: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21. In some embodiments, the population of expanded NK cells has been administered to a subject who has PD.

In some embodiments, the amount of expanded NK cells administered to a subject is a therapeutically effective amount.

In some embodiments, the therapeutically effective amount of expanded NK cells comprises 2×10⁹ to 9×10⁹ cells.

In some embodiments, IL-2 is added at a concentration of 50-1000 IU/mL during step ii).

In some embodiments, IL-21 is added at a concentration of 10-100 ng/mL during step ii).

In some embodiments, expansion of NK cells further comprises: co-culturing the at least one of CD56+ cells and/or CD3−/CD56+ cells with the combination of feeder cells, in the presence of IL-2 for a first period; and co-culturing the at least one of CD56+ cells and/or CD3−/CD56+ cells with the combination of feeder cells, in the presence of IL-21 for a second period.

In some embodiments, IL-21 is added more than once during Day 0-6 of the second period.

In some embodiments, IL-21 and the combination of feeder cells are added more than once during Day 0-6 of the second period.

In some embodiments, IL-21 is added more than once during the first six days of every fourteen-day cycle during the second period.

In some embodiments, the NK cells do not include a CAR.

In some embodiments, the NK cells do not include an engineered CAR.

In some embodiments, administration of the NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of the NK cells to a subject having Parkinson's disease improves the subject's mental sharpness/reaction by about 40% as compared to the subject's mental sharpness/reaction prior to administration of the NK cells.

In some embodiments, administration of the NK cells to a subject having Parkinson's disease improves the subject's fatigue/energy by about 40% as compared to the subject's fatigue/energy prior to administration of the NK cells.

In some embodiments, administration of the NK cells to a subject having Parkinson's disease improves the subject's speech by about 40% as compared to the subject's speech prior to administration of the NK cells.

In some embodiments, administration of the NK cells to a subject having Parkinson's disease improves the subject's left leg movement by about 30% as compared to the subject's left leg movement prior to administration of the NK cells.

In some embodiments, administration of the NK cells to a subject having Parkinson's disease improves the subject's left two finger stickiness by about 30% as compared to the subject's left two finger stickiness prior to administration of the NK cells.

In some embodiments, administration of the NK cells to a subject having Parkinson's disease improves the subject's face stiffness/smiling by about 30% as compared to the subject's face stiffness/smiling prior to administration of the NK cells.

In some embodiments, administration of the NK cells to a subject having Parkinson's disease improves the subject's depression by about 30% as compared to the subject's depression prior to administration of the NK cells.

In some embodiments, administration of the NK cells to a subject having Parkinson's disease improves the subject's neck stiffness by about 25% as compared to the subject's neck stiffness prior to administration of the NK cells.

In some embodiments, administration of the NK cells to a subject having Parkinson's disease improves the subject's left side weakness by about 25% as compared to the subject's mental left side weakness prior to administration of the NK cells.

In some embodiments, administration of the NK cells to a subject having Parkinson's disease improves the subject's left hand movement by about 20% as compared to the subject's left hand movement prior to administration of the NK cells.

In some embodiments, administration of the NK cells to a subject having Parkinson's disease improves the subject's balance by about 20% as compared to the subject's balance prior to administration of the NK cells.

In some embodiments, administration of about 4×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 6×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 8×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells to a subject having Parkinson's disease improves the subject's mental sharpness/reaction by about 40% as compared to the subject's mental sharpness/reaction prior to administration of the NK cells.

In some embodiments, administration of about 6×10⁹ or more NK cells to a subject having Parkinson's disease improves the subject's mental sharpness/reaction by about 40% as compared to the subject's mental sharpness/reaction prior to administration of the NK cells.

In some embodiments, administration of about 8×10⁹ or more NK cells to a subject having Parkinson's disease improves the subject's mental sharpness/reaction by about 40% as compared to the subject's mental sharpness/reaction prior to administration of the NK cells.

In some embodiments, administration of about 4×10⁹ or more NK cells to a subject having Parkinson's disease improves the subject's fatigue/energy by about 40% as compared to the subject's fatigue/energy prior to administration of the NK cells.

In some embodiments, administration of about 6×10⁹ or more NK cells to a subject having Parkinson's disease improves the subject's fatigue/energy by about 40% as compared to the subject's fatigue/energy prior to administration of the NK cells.

In some embodiments, administration of about 8×10⁹ or more NK cells to a subject having Parkinson's disease improves the subject's fatigue/energy by about 40% as compared to the subject's fatigue/energy prior to administration of the NK cells.

In some embodiments, administration of about 4×10⁹ or more NK cells to a subject having Parkinson's disease improves the subject's speech by about 40% as compared to the subject's speech prior to administration of the NK cells.

In some embodiments, administration of about 6×10⁹ or more NK cells to a subject having Parkinson's disease improves the subject's speech by about 40% as compared to the subject's speech prior to administration of the NK cells.

In some embodiments, administration of about 8×10⁹ or more NK cells to a subject having Parkinson's disease improves the subject's speech by about 40% as compared to the subject's speech prior to administration of the NK cells.

In some embodiments, administration of about 4×10⁹ or more NK cells to a subject having Parkinson's disease improves the subject's left leg movement by about 30% as compared to the subject's left leg movement prior to administration of the NK cells.

In some embodiments, administration of about 6×10⁹ or more NK cells to a subject having Parkinson's disease improves the subject's left leg movement by about 30% as compared to the subject's left leg movement prior to administration of the NK cells.

In some embodiments, administration of about 8×10⁹ or more NK cells to a subject having Parkinson's disease improves the subject's left leg movement by about 30% as compared to the subject's left leg movement prior to administration of the NK cells.

In some embodiments, administration of about 4×10⁹ or more NK cells to a subject having Parkinson's disease improves the subject's left two finger stickiness by about 30% as compared to the subject's left two finger stickiness prior to administration of the NK cells.

In some embodiments, administration of about 6×10⁹ or more NK cells to a subject having Parkinson's disease improves the subject's left two finger stickiness by about 30% as compared to the subject's left two finger stickiness prior to administration of the NK cells.

In some embodiments, administration of about 8×10⁹ or more NK cells to a subject having Parkinson's disease improves the subject's left two finger stickiness by about 30% as compared to the subject's left two finger stickiness prior to administration of the NK cells.

In some embodiments, administration of about 4×10⁹ or more NK cells to a subject having Parkinson's disease improves the subject's face stiffness/smiling by about 30% as compared to the subject's face stiffness/smiling prior to administration of the NK cells.

In some embodiments, administration of about 6×10⁹ or more NK cells to a subject having Parkinson's disease improves the subject's face stiffness/smiling by about 30% as compared to the subject's face stiffness/smiling prior to administration of the NK cells.

In some embodiments, administration of about 8×10⁹ or more NK cells to a subject having Parkinson's disease improves the subject's face stiffness/smiling by about 30% as compared to the subject's face stiffness/smiling prior to administration of the NK cells.

In some embodiments, administration of about 4×10⁹ or more NK cells to a subject having Parkinson's disease improves the subject's depression by about 30% as compared to the subject's depression prior to administration of the NK cells.

In some embodiments, administration of about 6×10⁹ or more NK cells to a subject having Parkinson's disease improves the subject's depression by about 30% as compared to the subject's depression prior to administration of the NK cells.

In some embodiments, administration of about 4×10⁹ or more NK cells to a subject having Parkinson's disease improves the subject's depression by about 30% as compared to the subject's depression prior to administration of the NK cells.

In some embodiments, administration of about 6×10⁹ or more NK cells to a subject having Parkinson's disease improves the subject's depression by about 30% as compared to the subject's depression prior to administration of the NK cells.

In some embodiments, administration of about 8×10⁹ or more NK cells to a subject having Parkinson's disease improves the subject's depression by about 30% as compared to the subject's depression prior to administration of the NK cells.

In some embodiments, administration of about 4×10⁹ or more NK cells to a subject having Parkinson's disease improves the subject's neck stiffness by about 25% as compared to the subject's neck stiffness prior to administration of the NK cells.

In some embodiments, administration of about 6×10⁹ or more NK cells to a subject having Parkinson's disease improves the subject's neck stiffness by about 25% as compared to the subject's neck stiffness prior to administration of the NK cells.

In some embodiments, administration of about 8×10⁹ or more NK cells to a subject having Parkinson's disease improves the subject's neck stiffness by about 25% as compared to the subject's neck stiffness prior to administration of the NK cells.

In some embodiments, administration of about 4×10⁹ or more NK cells to a subject having Parkinson's disease improves the subject's left side weakness by about 25% as compared to the subject's mental left side weakness prior to administration of the NK cells.

In some embodiments, administration of about 6×10⁹ or more NK cells to a subject having Parkinson's disease improves the subject's left side weakness by about 25% as compared to the subject's mental left side weakness prior to administration of the NK cells.

In some embodiments, administration of about 8×10⁹ or more NK cells to a subject having Parkinson's disease improves the subject's left side weakness by about 25% as compared to the subject's mental left side weakness prior to administration of the NK cells.

In some embodiments, administration of about 4×10⁹ or more NK cells to a subject having Parkinson's disease improves the subject's left hand movement by about 20% as compared to the subject's left hand movement prior to administration of the NK cells.

In some embodiments, administration of about 6×10⁹ or more NK cells to a subject having Parkinson's disease improves the subject's left hand movement by about 20% as compared to the subject's left hand movement prior to administration of the NK cells.

In some embodiments, administration of about 8×10⁹ or more NK cells to a subject having Parkinson's disease improves the subject's left hand movement by about 20% as compared to the subject's left hand movement prior to administration of the NK cells.

In some embodiments, administration of about 4×10⁹ or more NK cells to a subject having Parkinson's disease improves the subject's balance by about 20% as compared to the subject's balance prior to administration of the NK cells.

In some embodiments, administration of about 6×10⁹ or more NK cells to a subject having Parkinson's disease improves the subject's balance by about 20% as compared to the subject's balance prior to administration of the NK cells.

In some embodiments, administration of about 8×10⁹ or more NK cells to a subject having Parkinson's disease improves the subject's balance by about 20% as compared to the subject's balance prior to administration of the NK cells.

In some embodiments, administration of the NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 6×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 8×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

In some embodiments, administration of about 6×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

In some embodiments, administration of about 8×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

In some embodiments, administration of about 4×10⁹ or more NK cells to a subject having Parkinson's disease improves the subject's fatigue/energy by about 40% within about 48 hours of NK cell administration as compared to the subject's fatigue/energy prior to administration of the NK cells.

In some embodiments, administration of about 6×10⁹ or more NK cells to a subject having Parkinson's disease improves the subject's fatigue/energy by about 40% within about 48 hours of NK cell administration as compared to the subject's fatigue/energy prior to administration of the NK cells.

In some embodiments, administration of about 8×10⁹ or more NK cells to a subject having Parkinson's disease improves the subject's fatigue/energy by about 40% within about 48 hours of NK cell administration as compared to the subject's fatigue/energy prior to administration of the NK cells.

In some embodiments, administration of about 4×10⁹ or more NK cells to a subject having Parkinson's disease improves the subject's speech by about 40% within about 48 hours of NK cell administration as compared to the subject's speech prior to administration of the NK cells.

In some embodiments, administration of about 6×10⁹ or more NK cells to a subject having Parkinson's disease improves the subject's speech by about 40% within about 48 hours of NK cell administration as compared to the subject's speech prior to administration of the NK cells.

In some embodiments, administration of about 8×10⁹ or more NK cells to a subject having Parkinson's disease improves the subject's speech by about 40% within about 48 hours of NK cell administration as compared to the subject's speech prior to administration of the NK cells.

In some embodiments, administration of about 4×10⁹ or more NK cells to a subject having Parkinson's disease improves the subject's left leg movement by about 30% within about 48 hours of NK cell administration as compared to the subject's left leg movement prior to administration of the NK cells.

In some embodiments, administration of about 6×10⁹ or more NK cells to a subject having Parkinson's disease improves the subject's left leg movement by about 30% within about 48 hours of NK cell administration as compared to the subject's left leg movement prior to administration of the NK cells.

In some embodiments, administration of about 8×10⁹ or more NK cells to a subject having Parkinson's disease improves the subject's left leg movement by about 30% within about 48 hours of NK cell administration as compared to the subject's left leg movement prior to administration of the NK cells.

In some embodiments, administration of about 4×10⁹ or more NK cells to a subject having Parkinson's disease improves the subject's left two finger stickiness by about 30% within about 48 hours of NK cell administration as compared to the subject's left two finger stickiness prior to administration of the NK cells.

In some embodiments, administration of about 6×10⁹ or more NK cells to a subject having Parkinson's disease improves the subject's left two finger stickiness by about 30% within about 48 hours of NK cell administration as compared to the subject's left two finger stickiness prior to administration of the NK cells.

In some embodiments, administration of about 8×10⁹ or more NK cells to a subject having Parkinson's disease improves the subject's left two finger stickiness by about 30% within about 48 hours of NK cell administration as compared to the subject's left two finger stickiness prior to administration of the NK cells.

In some embodiments, administration of about 4×10⁹ or more NK cells to a subject having Parkinson's disease improves the subject's face stiffness/smiling by about 30% within about 48 hours of NK cell administration as compared to the subject's face stiffness/smiling prior to administration of the NK cells.

In some embodiments, administration of about 6×10⁹ or more NK cells to a subject having Parkinson's disease improves the subject's face stiffness/smiling by about 30% within about 48 hours of NK cell administration as compared to the subject's face stiffness/smiling prior to administration of the NK cells.

In some embodiments, administration of about 8×10⁹ or more NK cells to a subject having Parkinson's disease improves the subject's face stiffness/smiling by about 30% within about 48 hours of NK cell administration as compared to the subject's face stiffness/smiling prior to administration of the NK cells.

In some embodiments, administration of about 4×10⁹ or more NK cells to a subject having Parkinson's disease improves the subject's depression by about 30% within about 48 hours of NK cell administration as compared to the subject's depression prior to administration of the NK cells.

In some embodiments, administration of about 6×10⁹ or more NK cells to a subject having Parkinson's disease improves the subject's depression by about 30% within about 48 hours of NK cell administration as compared to the subject's depression prior to administration of the NK cells.

In some embodiments, administration of about 4×10⁹ or more NK cells to a subject having Parkinson's disease improves the subject's depression by about 30% within about 48 hours of NK cell administration as compared to the subject's depression prior to administration of the NK cells.

In some embodiments, administration of about 6×10⁹ or more NK cells to a subject having Parkinson's disease improves the subject's depression by about 30% within about 48 hours of NK cell administration as compared to the subject's depression prior to administration of the NK cells.

In some embodiments, administration of about 8×10⁹ or more NK cells to a subject having Parkinson's disease improves the subject's depression by about 30% within about 48 hours of NK cell administration as compared to the subject's depression prior to administration of the NK cells.

In some embodiments, administration of about 4×10⁹ or more NK cells to a subject having Parkinson's disease improves the subject's neck stiffness by about 25% within about 48 hours of NK cell administration as compared to the subject's neck stiffness prior to administration of the NK cells.

In some embodiments, administration of about 6×10⁹ or more NK cells to a subject having Parkinson's disease improves the subject's neck stiffness by about 25% within about 48 hours of NK cell administration as compared to the subject's neck stiffness prior to administration of the NK cells.

In some embodiments, administration of about 8×10⁹ or more NK cells to a subject having Parkinson's disease improves the subject's neck stiffness by about 25% within about 48 hours of NK cell administration as compared to the subject's neck stiffness prior to administration of the NK cells.

In some embodiments, administration of about 4×10⁹ or more NK cells to a subject having Parkinson's disease improves the subject's left side weakness by about 25% within about 48 hours of NK cell administration as compared to the subject's mental left side weakness prior to administration of the NK cells.

In some embodiments, administration of about 6×10⁹ or more NK cells to a subject having Parkinson's disease improves the subject's left side weakness by about 25% within about 48 hours of NK cell administration as compared to the subject's mental left side weakness prior to administration of the NK cells.

In some embodiments, administration of about 8×10⁹ or more NK cells to a subject having Parkinson's disease improves the subject's left side weakness by about 25% within about 48 hours of NK cell administration as compared to the subject's mental left side weakness prior to administration of the NK cells.

In some embodiments, administration of about 4×10⁹ or more NK cells to a subject having Parkinson's disease improves the subject's left hand movement by about 20% within about 48 hours of NK cell administration as compared to the subject's left hand movement prior to administration of the NK cells.

In some embodiments, administration of about 6×10⁹ or more NK cells to a subject having Parkinson's disease improves the subject's left hand movement by about 20% within about 48 hours of NK cell administration as compared to the subject's left hand movement prior to administration of the NK cells.

In some embodiments, administration of about 8×10⁹ or more NK cells to a subject having Parkinson's disease improves the subject's left hand movement by about 20% within about 48 hours of NK cell administration as compared to the subject's left hand movement prior to administration of the NK cells.

In some embodiments, administration of about 4×10⁹ or more NK cells to a subject having Parkinson's disease improves the subject's balance by about 20% within about 48 hours of NK cell administration as compared to the subject's balance prior to administration of the NK cells.

In some embodiments, administration of about 6×10⁹ or more NK cells to a subject having Parkinson's disease improves the subject's balance by about 20% within about 48 hours of NK cell administration as compared to the subject's balance prior to administration of the NK cells.

In some embodiments, administration of about 8×10⁹ or more NK cells to a subject having Parkinson's disease improves the subject's balance by about 20% within about 48 hours of NK cell administration as compared to the subject's balance prior to administration of the NK cells.

In some embodiments, any of the above steps can have further steps added between them. In some embodiments, any one or more of the above steps can be performed concurrently or out of the order provided herein.

### BRIEF DESCRIPTION OF THE DRAWINGS

FIG. 1 is a flow chart depicting some embodiments of a method for treating Parkinson's Disease in a subject in need thereof.

FIG. 2 is a graph depicting improvement from baseline (percentage) over time in a Parkinson's disease subject treated different amounts of NK cells.

FIG. 3 is a table showing questionnaire of a Movement Disorder Society-Sponsored Revision of the Unified Parkinson's Disease Rating Scale.

### DETAILED DESCRIPTION

Natural killer cells (NK cells) are one type of innate immune cells, which are known to non-specifically kill cancer, recognize and kill viruses, bacteria, and the like, and kill pathogens with enzymes such as perforin and granzyme or by Fas-FasL interaction. NK cells have also been reported to be able to kill activated T cells (Rabinovich B, et al., J Immunol, 2003: 170:3572-3576). This discovery is of interest in Parkinson's disease (PD) where elevated α-synuclein specific T cell responses have been detected prior to the onset of motor PD (Lindestam Arlehamn C, et al., Nature Comm., 2020: 11:1875-1886). Additionally, it has been reported that NK cells are capable of clearing α-synuclein within a mouse model of PD (Earls R, et al., PNAS, 2020: 117:1762-1771) and participating in nerve regeneration in the peripheral nervous system (Davies A, et al., Cell, 2019: 176:716-728). Accordingly, as disclosed herein, for PD, it is desirable to administer NK cells as a cellular therapy.

Parkinson's disease (PD) is one of the common neurodegenerative diseases that is characterized by selective degeneration of dopaminergic neurons in the substantia nigra, and misfolding of αa-synuclein into aggregates is thought to contribute to its pathology. Studies have shown that immune-inflammatory responses are involved in the development of PD and play an important role in α-synuclein scavenge. Natural killer (NK) cells are first responders in immune cells and can directly promote immune defense mechanisms by cytotoxicity and by secreting cytokines. Recent discoveries suggest that NK cells are increasingly recognized in the pathological features of PD. (Zhang et al., 2022) doi:10.3389/fnagi.2022.890816.

It has been reported that NK cells are capable of interacting with α-syn in animal models of PD and modulating neuroinflammation in neurodegenerative diseases (Poli et al., 2013; Earls et al., 2020; Garofalo et al., 2020). NK cells can efficiently internalize and degrade α-syn aggregates via the endosomal/lysosomal pathway. α-Syn aggregates attenuate NK cell cytotoxicity in a dose-dependent manner and decrease the release of the proinflammatory cytokine, IFN-γ. (Earls et al., 2020).

Without being bound by theory, NK cell depletion augments motor deficits implicating a protective role of NK cells in a PFF α-syn-induced mouse model of PD. The depletion of NK cells also exacerbates synuclein pathology and neuroinflammation in a PD mouse model. (Earls et al., 2020).

In some embodiments, SNK01 (an autologous NK cell therapy, as described herein) is administered to a PD subject to decrease the levels of α-syn and modulate neuroinflammation. In some embodiments, the levels of α-syn and neuroinflammation biomarkers are measured in the subject (e.g., before and/or after administering NK cells). In some embodiments, changes in MDS-UPDRS and/or NMSQ scores are assessed over time (e.g., before and/or after administering NK cells). In some embodiments, DAT imaging is used as a biomarker. In some embodiments, time to L-Dopa is used to assess outcome.

The therapeutic effect of NK cells in PD may be obtained using a large amount of NK cells having high purity, which can be obtained using a large amount of blood from the PD patient, where the proportion of NK cells in the blood can be small, only about 5 to 20%. Thus, without expansion, it can be difficult to use NK cells as an immunotherapeutic agent.

As a result, it is desirable to effectively expand and proliferate only the NK cells, but in a conventional method of proliferating NK cells, various expensive cytokines need to be used at a high concentration, thus the corresponding therapy is only available to some financially stable patients. Further, according to conventional methods of proliferating NK cells, other types (e.g., T cells, B cells, etc.) of immune cells may be present together with the NK cells, and allogeneic administration of the NK cells containing T cells may cause a graft versus host disease (GVHD) and allogeneic administration of the NK cells containing B cells to blood-type incompatible subjects may cause a passenger B-lymphocyte syndrome, and thus, the therapeutic effect in PD is not maximized.

Further, in addition to expanding and proliferating NK cells, it is desirable to highly maintain the functions of NK cells until the expanded and proliferated NK cells are actually used. As a result, the development of a composition capable of promoting the proliferation of the NK cells, increasing production of cytokines such as TNFα, INFγ and GM-CSF derived from the NK cells, and increasing activity of the NK cells is sought.

Earls et al have recently reported that NK cells clear α-synuclein and the depletion of NK cell exacerbates synuclein pathology in a mouse model of α-synucleinopathy, in which they used NK-92 NK cell lines and primary human NK cells from healthy donors. The result showed that both NK92 and primary NK cells internalize α-synuclein through TLR-4 and TLR-2 and degrade α-synuclein. Since SNK is a culture-expanded and activated NK cells, it is appreciated herein that these cell likely have more ability to internalize and degrade α-synuclein than NK-92 and primary NK cells.

Provided herein are methods for treating PD comprising administering high-purity natural killer cells.

### Terminology

All terms are to be given their ordinary and customary meaning as understood by one of ordinary skill in the art, in view of the present disclosure.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The ranges disclosed herein also encompass any and all overlap, sub-ranges, and combinations thereof. Language such as “up to,” “at least,” “greater than,” “less than,” “between,” and the like includes the number recited.

Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The term “generally” as used herein represents a value, amount, or characteristic that predominantly includes or tends toward a particular value, amount, or characteristic. As an example, in certain embodiments, the term “generally uniform” refers to a value, amount, or characteristic that departs from exactly uniform by less than 20%, less than 15%, less than 10%, less than 5%, less than 1%, less than 0.1%, and less than 0.01%.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about 5.0 cm” includes “5.0 cm.”

In some embodiments, the cell therapeutic composition may include a therapeutically effective amount of cell therapeutic agent for treatment of diseases. The term “therapeutically effective amount” means an amount of an active ingredient or a cell therapeutic composition which induces biological or medical responses in tissue systems, animals, or humans which are considered by researchers, veterinarians, physicians, or other clinicians, and includes an amount of inducing alleviation of symptoms of diseases or disorders to be treated. It will be apparent to those skilled in the art that the cell therapeutic agent included in the cell therapeutic composition may be changed according to a desired effect. Therefore, the optimal content of the cell therapeutic agent may be easily determined by those skilled in the art, and may be adjusted according to various factors including a type of disease, severity of the disease, contents of other ingredients contained in the composition, a type of formulation, and an age, a weight, a general health condition, a gender, and a diet of a patient, an administration time, an administration route, a secretion ratio of the composition, a treatment period, and simultaneously used drugs. It is important to include an amount capable of obtaining a maximum effect by a minimum amount without side effects by considering all of the factors. For example, in some embodiments, the cell therapeutic composition may include a cell therapeutic agent of 1×10⁶ to 5×10⁸ cells per kg of body weight.

As used herein, the term “Parkinson's Disease” (PD) refers to a progressive disorder that affects the nervous system and the parts of the body controlled by the nerves. Symptoms start slowly. The first symptom may be a barely noticeable tremor in just one hand. Tremors are common, but the disorder may also cause stiffness or slowing of movement. Parkinson's disease signs and symptoms can be different for everyone. Early signs may be mild and go unnoticed. Symptoms often begin on one side of the body and usually remain worse on that side, even after symptoms begin to affect the limbs on both sides. Parkinson's signs and symptoms may include: a tremor, or rhythmic shaking, slowed movement (bradykinesia), impaired posture and balance, loss of automatic movements, speech changes, and/or writing changes.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

In some embodiments, a method of treating PD in a subject is provided. In some embodiments, the method comprises: identifying a subject, wherein the subject has PD; and administering to the subject an expanded natural killer (NK) cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a method of cell therapy is provided, comprising: identifying a subject, wherein the subject has PD; and administering to the subject an expanded NK cell population. In some embodiments, the NK cells are expanded by a method comprising: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21.

In some embodiments, a population of expanded NK cells is provided. In some embodiments, the NK cells were expanded by a method that comprises: i) isolating at least one of CD56+ cells and/or CD3−/CD56+ cells from the PBMCs; ii) co-culturing the at least one of CD56+ cells and/or CD3−/CD56+ cells with a combination of feeder cells in the presence of at least two cytokines; iii) wherein the combination of feeder cells comprises irradiated Jurkat cells and irradiated Epstein-Barr virus transformed lymphocyte continuous line (EBV-LCL) cells; and iv) wherein the at least two cytokines comprise IL-2 and IL-21. In some embodiments, the population of expanded NK cells has been administered to a subject who has PD.

In some embodiments, a method of treating Parkinson's disease (PD) in a subject is provided. In some embodiments, the method comprises identifying a subject, wherein the subject has PD; and administering to the subject a therapeutically effective amount of an autologous natural killer cell (NK) cell population.

In some embodiments, the amount of expanded NK cells administered to a subject is a therapeutically effective amount.

In some embodiments, the therapeutically effective amount of expanded NK cells comprises 2×10⁹ to 9×10⁹ cells. In some embodiments, the amount is 0.1×10⁹, 0.5×10⁹, 1×10⁹, 2×10⁹, 3×10⁹, 4×10⁹, 5×10⁹, 6×10⁹, 7×10⁹, 8×10⁹, 9×10⁹, 10×10⁹, 11×10⁹, 12×10⁹, or more. In some embodiments, the therapeutically effective amount of expanded NK cells comprises 0.1×10⁹-1×10¹² cells, 0.5×10⁹-1×10¹¹ cells, 1×10⁹-1×10¹⁰ cells, 1×10⁹-1×10¹¹ cells, or 1×10⁹-5×10¹⁰ cells. In some embodiments it is any one of the preceding amounts given every 1, 2, 3, 4, 5, 6, 7, or 8 weeks. In some embodiments, it is 3-5 billion cells given every 2-4 weeks (e.g., 4 billion cells every 3 weeks). In any method of the present disclosure, in some embodiments, the maximum amount of expanded NK cells is 9×10⁹ cells. In any method of the present disclosure, in some embodiments, the maximum amount of expanded NK cells is 1×10¹² cells.

In some embodiments, IL-2 is added at a concentration of 50-1000 IU/mL during step ii).

In some embodiments IL-21 is added at a concentration of 10-100 ng/mL during step ii).

In some embodiments, expansion of NK cells further comprises: co-culturing the at least one of CD56+ cells and/or CD3−/CD56+ cells with the combination of feeder cells, in the presence of IL-2 for a first period; and co-culturing the at least one of CD56+ cells and/or CD3−/CD56+ cells with the combination of feeder cells, in the presence of IL-21 for a second period.

In some embodiments, IL-21 is added more than once during Day 0-6 of the second period.

In some embodiments, IL-21 and the combination of feeder cells are added more than once during Day 0-6 of the second period.

In some embodiments, IL-21 is added more than once during the first six days of every fourteen-day cycle during the second period.

In some embodiments, the NK cells do not include a chimeric antigen receptor (CAR).

In some embodiments, the NK cells do not include an engineered CAR.

In any method of the present disclosure, in some embodiments, the NK cells to be administered can be NK cells that have been expanded with any suitable option for expanding NK cells. In some embodiments, the NK cells are autologous (e.g., autologous to the subject to which the NK cells are administered). In some embodiments, the NK cells are or comprise SNK01. (See www(dot)sec(dot)gov/ix?doc=/Archives/edgar/data/1845459/000110465923074785/gfor-20230331xs4a.htm, which is incorporated by reference herein as to SNK01). As used herein, “SNK01” denotes SNK01 autologous NK cells produced by NKGen Biotech, Inc. (Irvine, CA). In some embodiments, the NK cells are or comprise SNK01 autologous cells, produced by NKGen Biotech, Inc. (Irvine, CA). Suitable options for expanding NK cells are provided in, e.g., PCT publication No. WO 2019/152663, which is incorporated by reference in its entirety herein. In some embodiments, the NK cells are allogeneic (e.g., allogeneic to the subject to which the NK cells are administered).

In some embodiments, any of the above steps can have further steps added between them. In some embodiments, any one or more of the above steps can be performed concurrently or out of the order provided herein.

A method for producing high-purity NK cells without using expensive cytokines has been developed. After CD56+ cells are isolated from peripheral blood mononuclear cells, when the CD56+ cells isolated from peripheral blood mononuclear cells are co-cultured with feeder cells in the presence of cytokines, high-purity CD56+NK cells could be produced. Also, a cell therapeutic composition for treating PD comprising NK cells which are effectively usable for autologous and allogeneic therapy is provided herein. As a result, when a specific cytokine was added to CD56+NK cells isolated from peripheral blood mononuclear cells, high survival rate and high activity were exhibited. Therefore, in some embodiments, the treatment of PD involves or includes a method for expanding NK cells and to provide a cell therapeutic composition for the treatment of PD comprising expanded peripheral blood-derived CD56+NK cells.

According to some embodiments, a method for producing high-purity NK cells may include: isolating peripheral blood mononuclear cells (PBMCs) from a blood sample (“First Isolation Step”); isolating cells selected from a group consisting of CD56+ cells and CD3−/CD56+ cells from the peripheral blood mononuclear cells (“Second Isolation Step”); and co-culturing the cells selected from a group consisting of CD56+ cells and CD3−/CD56+ cells together with feeder cells in the presence of cytokine (“Culturing Step”). Each step is described in greater detail herein. The CD3−/CD56+ cells produced according to the disclosed method may exhibit not only higher purity and higher activity, but also other distinguished characteristics, such as having different surface markers or activated receptors, for example, one or more from CD16, CD25, CD27, CD28, CD69, CD94/NKG2C, CD94/NKG2E, CD266, CD244, NKG2D, KIR2S, KIR3S, Ly94D, NCRs, IFN-a, IFN-b,CXCR3, CXCR4, CX3CR1, CD62L and CD57, as compared with NK cells produced from peripheral blood mononuclear cells without isolating CD56+ cells.

### First Isolation Step

In the present specification, the “blood sample” may be, but not limited to, whole blood of the peripheral blood or leukocytes isolated from the peripheral blood using leukapheresis. Further, the peripheral blood may be obtained from a normal person, a patient having a risk of PD, or a PD patient, but the source of the peripheral blood is not limited thereto.

In the present specification, the term “leukapheresis” may refer to a method of selectively removing (isolating) leukocytes from the collected blood and then giving the blood to a patient again, and in some embodiments, the leukocytes isolated by the method may be used without additional methods such as a Ficoll-Hypaque density gradient method.

In the present specification, the term “peripheral blood mononuclear cell” may be used interchangeably with “PBMC”, “mononuclear cell” or “monocyte”, and may refer to a mononuclear cell isolated from the peripheral blood which is generally used for anti-PD immunotherapy. The peripheral blood mononuclear cells may be obtained from the collected human blood using known methods such as a Ficoll-Hypaque density gradient method.

In some embodiments, the peripheral blood mononuclear cells may be autologous, but allogeneic peripheral blood mononuclear cells may also be used for producing high-purity NK cells for immunotherapy according to methods described herein. Further, in some embodiments, the peripheral blood mononuclear cells may be obtained from a normal person, but the peripheral blood mononuclear cells may be also obtained from a patient having a risk of PD and/or a PD patient.

In the present specification, the term “CD56+ cells” may be used interchangeably with “CD56+ NK cells”, or “CD56+ natural killer cells”, and the term “CD3−/CD56+ cells” may be used interchangeably with “CD3−/CD56+NK cells.” The CD56+ cells or CD3−/CD56+ cells may include cells in which CD56 glycoprotein on the cell surface is expressed, or further, cells in which CD3 glycoprotein is not expressed while the CD56 glycoprotein is expressed. Even the same type of immune cells may have differences in CD type attached to the cell surface and expression rate and thus, the functions thereof may be different.

### Second Isolation Step

In some embodiments, the isolating of the CD56+ natural killer cells from the blood sample may be performed by an isolating method using at least one selected from the group consisting of CD56 microbeads and CD3 microbeads, or an isolating method using equipment such as CliniMACSs, a flow cytometry cell sorter, etc.

For example, the isolating method using the CD56 microbeads and/or the CD3 microbeads may be performed by adding the CD56 microbeads to PBMCs and then removing non-specific binding, or performed by adding the CD3 microbeads to the PBMCs to remove specific binding and then adding the CD56 microbeads again to remove non-specific binding. In some instances, through isolating CD56+ cells and/or CD3−/CD56+ cells from PBMCs, T cells or other non-natural killer cells may be removed.

### Culturing Step

In the present specification, the term “cytokine” may refer to an immunoactive compound that is usable to induce the peripheral blood mononuclear cells to differentiate into NK cells.

In some embodiments, the cytokine may be interleukin-2 (IL-2), IL-15, IL-21, FMS-like tyrosine kinase 3 ligand (Flt3-L), a stem cell factor (SCF), IL-7, IL-18, IL-4, type I interferons, a granulocyte-macrophage colony-stimulating factor (GM-CSF), and an insulin-like growth factor 1 (IGF 1), but not limited thereto.

In some embodiments, the cytokine may be used at a concentration of 50-1,000, 50-900, 50-800, 50-700, 50-600, 50-550, 100-550, 150-550, 200-550, 250-550, 300-550, 350-550, 400-550, 450-550 IU/mL. Conventional methods of proliferating NK cells utilize high concentrations of various cytokines. Conversely, in some embodiments of the method of proliferating NK cells described herein, since two types of feeder cells may be used with the high-purity CD56+ cells, NK cells with high yield and high purity may be proliferated using only low concentrations of one cytokine.

In the present specification, the term “feeder cell” may refer to a cell that does not divide and proliferate, but has metabolic activity to produce various metabolites and thus, helps the proliferation of target cells.

In some embodiments, the feeder cells may be at least one selected from the group consisting of irradiated Jurkat cells, irradiated Epstein-Barr virus transformed lymphocyte continuous line (EBV-LCL) cells, and PBMC, HFWT, RPMI 1866, Daudi, MM-170, K562 or cells genetically modified by targeting K562 (for example, K562-mbIL-15-41BB ligand). For example, in one embodiment, the feeder cells may be the irradiated Jurkat cells and the EBV-LCL cells.

In the present specification, the term “Jurkat cell” or “Jurkat cell line” may refer to a blood cancer (immortalized acute T cell leukemia) cell line, which has been developed by Dr. Arthur Weiss of the University of California at San Francisco. Jurkat cells, in which various chemokine receptors are expressed and capable of producing IL-2, have not generally been considered as a possible candidate of the feeder cells for immunotherapy because MHC class I, which is a natural killer cell activation inhibitor, is highly expressed on the cell surface thereof. The Jurkat cells may be obtained from the ATCC (ATCC TIB-152).

In the present specification, the term “EBV-LCL cell” or “EBV-LCL cell line” refers to an Epstein-Barr virus transformed lymphocyte continuous line (EBV-LCL) (D. M. Koelle et al., J Clin Invest, 1993: 91:961-968), which is a B cell line that is made by infecting human B cells with Epstein-Barr virus in a test tube. The EBV-LCL cells may be directly prepared and used in a general laboratory by a method of adding cyclosporine A in a process of infecting EBV in the PBMC. In some embodiments, the EBV-LCL cell may be prepared by following steps. 30×10⁶ PBMCs are added in 9 mL of a culture medium, the mixture is added in a T 25 culture flask, and then 9 mL of an EBV supernatant is added. 80 μL of cyclosporine A (50 μg/mL) is added and then cultured at 37° C. After 7 days of culture, a half of supernatant is removed, a fresh culture medium is added, and then 40 μL of cyclosporine A is added. The same process may be repeated once every 7 days until 28 days of culture. The cell line may be usable after 28 days of culture, and from this time, the cell line may be cultured in the culture medium without adding cyclosporine A.

The Jurkat cells and the EBV-LCL cells may be used as the feeder cells after irradiation. In some embodiments, the irradiated Jurkat cells and the irradiated EBV-LCL cells may be included at a content ratio of 1:0.1-5, 1:0.1-4, 1:0.1-3, 1:0.1-2, 1:0.1-1.5, 1:0.5-1.5, 1:0.75-1.25, 0.1-5:1, 0.1-4:1, 0.1-3:1, 0.1-2:1, 0.1-1.5:1, 0.5-1.5:1 or 0.75-1.25:1. For example, the irradiated Jurkat cells and the irradiated EBV-LCL cells may be included at a content ratio of 1:1.

In some embodiments, the irradiated Jurkat cells and the irradiated EBV-LCL cells may be obtained by treating with irradiation of 50-500, 50-400, 50-300, 50-200, 50-150, 70-130, 80-120 or 90-110 Gy. For example, the irradiated Jurkat cells and/or the irradiated EBV-LCL cells may be obtained by treating Jurkat cells and/or EBV-LCL cells with irradiation of 100 Gy.

In some embodiments, the culturing may be performed for 1-50, 1-42, 1-40, 1-35, 1-20, 1-19, 1-18, 1-17, 1-16, 1-15 or 1-14 days.

In some embodiments, the culturing step may further include following steps: co-culturing with the feeder cells and a first cytokine (“first culturing step”); and further co-culturing after addition of a second cytokine (“second culturing step”)

The second culturing step may include adding the second cytokine once or more between day 0-6 of culturing. For example, the second culturing step may include adding the second cytokine once on each of day 0 and day 3 of culturing.

The second culturing step may include adding the second cytokine and the feeder cells during the first 6 days of the cycle of 14 days of culturing. For example, the second culturing step may include adding the feeder cells during a 14 days cycle, and adding the second cytokine on day 3 and 6 of each cycle once each.

In some embodiments, the first cytokine may be IL-2. In some embodiments, the second cytokine may be IL-21. In some embodiments, the second cytokine may be used at the concentration of 10-1000, 10-500, 10-100, 20-100, 30-100, 40-100, 50-100 or 10-50 ng/mL. In some embodiments, culturing with the addition of the second cytokine once or more during day 0-6 may exhibit superior proliferation and/or activity. In some embodiments, culturing with the addition of the feeder cells and the second cytokine for six days in the cycle of 14 days may exhibit superior proliferation and/or activity.

In some embodiments, the co-culturing may be performed by including the peripheral blood mononuclear cells and the feeder cells (for example, the Jurkat cells and the EBV-LCL cells) at a mixing ratio of 1:1-100, 1:1-90, 1:1-80, 1:1-70, 1:10-65, 1:20-65, 1:30-65, 1:40-65, 1:50-65 or 1:55-65.

The co-culturing may be performed in a medium and any suitable media generally used for induction and proliferation of the peripheral blood mononuclear cells to the NK cells in the art may be used without a limitation as such a medium. For example, an RPMI-1640, DMEM, x-vivo10, x-vivo20, or cellgro SCGM medium may be used as such a medium. In addition, the culture conditions such as a temperature may follow any suitable culture conditions of the peripheral blood mononuclear cells known in the art.

In some embodiments, within the produced NK cells, a ratio or purity of the CD56+ NK cells may be 85% or more, 90% or more, or 95% or more, or 98% or more with respect to the whole cells. In some embodiments, within the produced NK cells, a ratio of T cells to whole cells may be 15% or less, 10% or less, 5% or less, 2% or less, 1% or less.

In some embodiments, the cytokines IL-2 and IL-21 are capable of supporting expansion of a CD3−/CD56+, or CD56+ population in vitro. In some embodiments, the population of CD3−/CD56+or CD56+ cells expanded with IL-2 and IL-21 possesses an NK cell phenotype.

In some embodiments, the method of treatment of PD involves culturing and/or expanding cells in line with one or more of the approaches outlined in U.S. Pat. No. 10,590,385, which is incorporated herein by reference in its entirety.

In the present specification, the term “peripheral blood-derived” may mean that the cells are derived from “whole blood of the peripheral blood” or “leukocytes isolated from the peripheral blood using leukapheresis.” The peripheral blood derived CD56+ NK cells may be used interchangeably with peripheral blood mononuclear cell (PBMC) derived CD56+ NK cells.

In some embodiments, the term “subject” refers to a mammal which is a subject for treatment, observation, or testing, and preferably, a human. The subject may be a patient of PD, but not limited thereto.

In some embodiments, in the case of an adult, the cell therapeutic composition may be administered once to several times a day. The cell therapeutic composition may be administered every day or in a 2-180 day interval. the cell therapeutic agent included in the composition may include 1×10⁶ to 1×10¹¹ peripheral blood-derived CD56+ natural killer cells, for example, about 1×10⁶ to 1×10⁸ NK cells per kg of body weight. In some preferred embodiments, the cell therapeutic agent included in the composition may include 2×10⁹ to 9×10⁹ peripheral blood-derived CD56+ natural killer cells. In some embodiments, the peripheral blood-derived CD56+ natural killer cells in the cell therapeutic composition are at least about 90% pure. In some embodiments, the cytokine is IL-2 at a concentration ranging from about 50-50,000 IU/ml.

In some embodiments, the cell therapeutic composition of the present invention may be administered by any suitable method, such as administration through a rectal, intravenous, intraarterial, intraperitoneal, intramuscular, intrasternal, percutaneous, topical, intraocular, or intradermal route. In some embodiments, the NK cells included in the composition may be allogeneic, i.e. obtained from a person other than the subject being treated. In some embodiments, the person may be a normal person or a patient with PD. In some embodiments, the NK cells included in the composition may be autologous, i.e. obtained from the subject being treated.

In some embodiments the subject has Parkinson's disease (PD). PD is a neurodegenerative disorder that affects the central nervous system (CNS). PD is characterized by abnormal aggregations of misfolded α-synuclein protein into Lewy bodies within the brain. Hallmarks of PD include motor deficits such as tremors and difficulty moving, and cognitive problems including depression and dementia.

In some embodiments, identifying a subject with PD comprises a medical diagnosis of PD. In some embodiments, diagnosis of PD comprises assessment of slowness of movement (bradykinesia) in addition to evaluation of either rigidity, resting tremor, or postural instability. In some embodiments, diagnosis of PD comprises imaging via CAT, MRI, PET, and/or DaT scan.

Natural killer cells (NK cells) are one type of innate immune cells, which are known to non-specifically kill cancer, recognize and kill viruses, bacteria, and the like, and kill pathogens with enzymes such as perforin and granzyme or by Fas-FasL interaction.

The protein α-synuclein is found primarily in the axons of presynaptic neurons within the human brain. α-synuclein is known to aggregate into insoluble fibrils within Lewy bodies. The presence of Lewy bodies is a characteristic feature of PD. Aggregated α-synuclein is known to exert a neurotoxic effect contributing to neuronal death.

In some embodiments the administered expanded NK cells are capable of clearing away aggregated α-synuclein in the CNS. In some embodiments, NK cell mediated removal of α-synuclein aggregates decreases neurotoxicity and neuronal death. In some embodiments, the NK cell mediated reduction in aggregated α-synuclein exerts a therapeutic effect in PD.

In some embodiments the NK cells administered to the patient are autologous to the subject. In some embodiments, the NK cells administered to the patient are allogeneic with respect to the subject. In some embodiments, the NK cells administered are derived from a healthy subject. In some embodiments, the NK cells administered are derived from a subject, with disease such as a subject with PD.

In some embodiments, the NK cell population has undergone expansion prior to administration. In some embodiments, an autologous NK cell population was expanded in vitro prior to administration. In some embodiments, an allogeneic NK cell population was expanded in vitro prior to administration. In some embodiments NK cell expansion is accomplished by feeder cells. In some embodiments, NK cell expansion is accomplished by cytokine stimulation. In some embodiments, NK cell expansion is accomplished by both cytokines and feeder cells. In some embodiments, expansion of NK cells results in a population with a high purity of NK cells.

In some embodiments, the ratio of CD56+ NK cells to whole cells (purity) may be 85% or more, 90% or more, 95% or more, or 98% or more.

In some embodiments, the composition may not include T cells, or may include only trace amount of T cells. For example, the ratio of T cells to whole cells in the composition may be less than 15%, less than 10%, less than 5%, less than 2%, less than 1% or less.

In some embodiments, the NK cells are co-administered with a cytokine. In some embodiments the cytokine is IL-2, IL-21, IL-15, Flt3-L, IL-7, SCF, IL-18, IL-4, type I IFN, GM-CSF, IGF I, or any combinations thereof. In some embodiments, the cytokine may be used at a concentration of 18-180,000, 20-100,000, 50-50,000, 50-1,000, 50-900, 50-800, 50-700, 50-600, 50-550, 100-550, 150-550, 200-550, 250-550, 300-550, 350-550, 400-550, 450-550 IU/mL. When the cytokine is used in these ranges, it may suppress apoptosis of the NK cells included in the treatment composition and increase activity of the NK cells.

In the present specification, the term “cell therapeutic agent” refers to a medicine which is used for treatment, diagnosis, and prevention through a series of actions, such as proliferating and screening autologous, allogeneic, and xenogeneic living cells in vitro for restoring functions of cells and tissues or changing biological characteristics of the cells by other methods. The cell therapeutic agents have been regulated as medical products from 1993 in USA and 2002 in Korea. These cell therapeutic agents may be largely classified into two fields, that are, first, stem cell therapeutic agents for tissue regeneration or recovery of organ functions, and second, immune cell therapeutic agents for regulation of immune responses, such as inhibition of the immune response or enhancement of the immune response in vivo.

The cell therapeutic composition described herein may be formulated in a suitable form together with a pharmaceutically acceptable carrier suitable or generally used for cell therapy. The “pharmaceutically acceptable” refers to a composition which is physiologically acceptable and does not generally cause an allergic reaction such as gastrointestinal disorders, dizziness, or the like, or similar reactions thereto, when being administered to the human body. The pharmaceutically acceptable carrier may include, for example, parenteral administration carries such as water, suitable oils, saline, aqueous glucose and glycol, and the like, and further include stabilizers and preservatives. The suitable stabilizer includes an antioxidant such as sodium hydrogen sulfite, sodium sulfite, or ascorbic acid, sucrose, albumin, or the like. The suitable preservative includes DMSO, glycerol, ethylene glycol, sucrose, trehalose, dextrose, polyvinylpyrrolidone, or the like.

The cell therapeutic composition may also be administered by any device in which the cell therapeutic agent may move to the target cell.

In some embodiments, the NK cells are not engineered to express a T cell receptor (TCR) or CAR. In some embodiments, the NK cells are not engineered to express additional stimulatory or co-stimulatory domains. In some embodiments, the NK cells are not engineered to express an antigen binding domain. In some embodiments, the NK cells are not engineered to express additional members of the NKG2 family. In some embodiments, the NK cells are expanded without additional engineering steps.

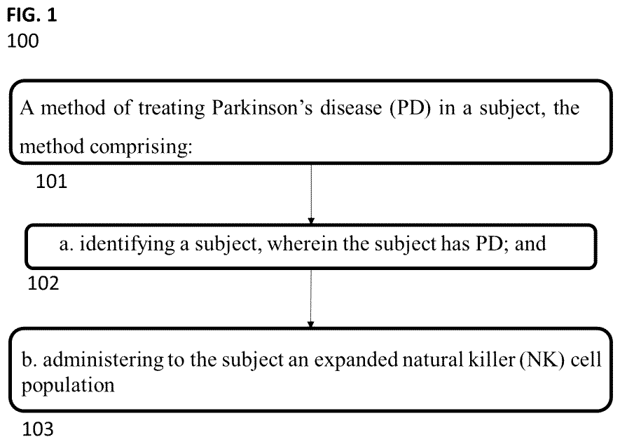

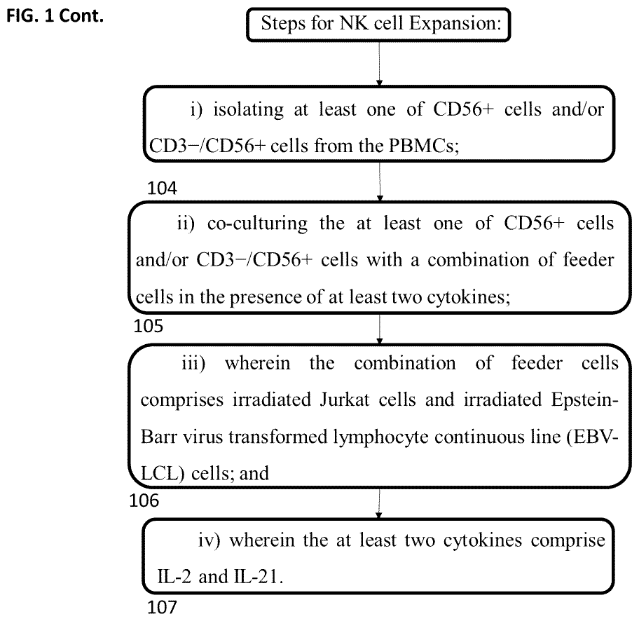

FIG. 1 is a flow chart depicting some embodiments of a method for treating Parkinson's Disease in a subject in need thereof.

In some embodiments, a method of treating Parkinson's disease (PD) in a subject 101 is disclosed. In some embodiments, the method comprises identifying a subject, wherein the subject has PD 102; and administering to the subject an expanded natural killer (NK) cell population 103. In some embodiments, the NK cells are expanded by a method comprising: i) isolating at least one of CD56+ cells and/or CD3−/CD56+ cells from the PBMCs 104; ii) co-culturing the at least one of CD56+ cells and/or CD3−/CD56+ cells with a combination of feeder cells in the presence of at least two cytokines 105; iii) wherein the combination of feeder cells comprises irradiated Jurkat cells and irradiated Epstein-Barr virus transformed lymphocyte continuous line (EBV-LCL) cells 106; and iv) wherein the at least two cytokines comprise IL-2 and IL-21 107. In some embodiments, administration of the NK cells to a subject having PD improves one or more symptoms of PD in the subject. For example, in some embodiments, administration of the NK cells improves the subject's mental sharpness/reaction, fatigue/energy, speech, left leg movement, left two finger stickiness, face stiffness/smiling, depression, neck stiffness, left side weakness, left hand movement, and/or balance, or any combination thereof, as compared to the subject's baseline mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. In some embodiments, administration of the NK cells improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by about 1, 2, 3, 4, 5, 6, 7, 8, 9, 10, 20, 25, 30, 40, 50, 60, 75, 80, 90, 100, 150, 200, 250, 300, 400, or 500%, or by a percentage that is in a range defined by any two of the preceding values, as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. For example, in some embodiments, administration of the NK cells improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 1-500, 1-250, 1-100, 1-75, 1-50, 1-25, 1-20, 20-500, 20-250, 20-100, 20-75, 20-50, 20-40, 20-30, 20-25, 25-500, 25-250, 25-100, 25-75, 25-50, 25-40, 25-30, 30-500, 30-250, 30-100, 30-75, 30-50, 30-40, 40-500, 40-250, 40-100, 40-75, 40-50, 50-500, 50-250, 50-100, 50-75, 75-500, 75-250, 75-100, 100-500, 100-250, or 250-500%, as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. In some embodiments, administration of the NK cells continues until the subject's mental sharpness/reaction, fatigue/energy, speech, left leg movement, left two finger stickiness, face stiffness/smiling, depression, neck stiffness, left side weakness, left hand movement, and/or balance, or any combination thereof, improves as compared to the subject's baseline mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. In some embodiments, administration of the NK cells continues until the subject's mental sharpness/reaction, fatigue/energy, speech, left leg movement, left two finger stickiness, face stiffness/smiling, depression, neck stiffness, left side weakness, left hand movement, and/or balance, or any combination thereof, improves by about 1, 2, 3, 4, 5, 6, 7, 8, 9, 10, 20, 25, 30, 40, 50, 60, 75, 80, 90, 100, 150, 200, 250, 300, 400, or 500%, or by a percentage that is in a range defined by any two of the preceding values, as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. For example, in some embodiments, administration of the NK cells continues until the subject's mental sharpness/reaction, fatigue/energy, speech, left leg movement, left two finger stickiness, face stiffness/smiling, depression, neck stiffness, left side weakness, left hand movement, and/or balance, or any combination thereof, improves by between about 1-500, 1-250, 1-100, 1-75, 1-50, 1-25, 1-20, 20-500, 20-250, 20-100, 20-75, 20-50, 20-40, 20-30, 20-25, 25-500, 25-250, 25-100, 25-75, 25-50, 25-40, 25-30, 30-500, 30-250, 30-100, 30-75, 30-50, 30-40, 40-500, 40-250, 40-100, 40-75, 40-50, 50-500, 50-250, 50-100, 50-75, 75-500, 75-250, 75-100, 100-500, 100-250, or 250-500%, as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells. The therapeutic effect of administering the NK cells according the any embodiment provided herein can be measured using any suitable option. In some embodiments, the therapeutic effect of administering the NK cells is determined using a PD rating scales NMSQ (Non-Motor Symptoms Questionnaire) and/or UPDRS or the Movement Disorder Society-Sponsored Revision of the MDS (MSD-UPDRS). In some embodiments, the therapeutic effect of administering the NK cells is determined by self-assessment of cognitive impairment, fatigue, speech, facial expression, rigidity in the neck, finger tapping in the left hand, hand movements of the left hand, agility of the left leg, and/or posture stability by the subject, e.g., using a PD rating scales NMSQ (Non-Motor Symptoms Questionnaire). In some embodiments, the therapeutic effect of administering the NK cells is determined using one or more imaging techniques, such as, without limitation, DaTscan imaging.

In some embodiments, administration of about 4×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 6×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 8×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells (e.g., expanded NK cells) to a subject having Parkinson's disease improves the subject's mental sharpness/reaction by about 40% as compared to the subject's mental sharpness/reaction prior to administration of the NK cells.

In some embodiments, administration of about 6×10⁹ or more NK cells (e.g., expanded NK cells) to a subject having Parkinson's disease improves the subject's mental sharpness/reaction by about 40% as compared to the subject's mental sharpness/reaction prior to administration of the NK cells.

In some embodiments, administration of about 8×10⁹ or more NK cells (e.g., expanded NK cells) to a subject having Parkinson's disease improves the subject's mental sharpness/reaction by about 40% as compared to the subject's mental sharpness/reaction prior to administration of the NK cells.

In some embodiments, administration of about 4×10⁹ or more NK cells (e.g., expanded NK cells) to a subject having Parkinson's disease improves the subject's fatigue/energy by about 40% as compared to the subject's fatigue/energy prior to administration of the NK cells.

In some embodiments, administration of about 6×10⁹ or more NK cells (e.g., expanded NK cells) to a subject having Parkinson's disease improves the subject's fatigue/energy by about 40% as compared to the subject's fatigue/energy prior to administration of the NK cells.

In some embodiments, administration of about 8×10⁹ or more NK cells (e.g., expanded NK cells) to a subject having Parkinson's disease improves the subject's fatigue/energy by about 40% as compared to the subject's fatigue/energy prior to administration of the NK cells.

In some embodiments, administration of about 4×10⁹ or more NK cells (e.g., expanded NK cells) to a subject having Parkinson's disease improves the subject's speech by about 40% as compared to the subject's speech prior to administration of the NK cells.

In some embodiments, administration of about 6×10⁹ or more NK cells (e.g., expanded NK cells) to a subject having Parkinson's disease improves the subject's speech by about 40% as compared to the subject's speech prior to administration of the NK cells.

In some embodiments, administration of about 8×10⁹ or more NK cells (e.g., expanded NK cells) to a subject having Parkinson's disease improves the subject's speech by about 40% as compared to the subject's speech prior to administration of the NK cells.

In some embodiments, administration of about 4×10⁹ or more NK cells (e.g., expanded NK cells) to a subject having Parkinson's disease improves the subject's left leg movement by about 30% as compared to the subject's left leg movement prior to administration of the NK cells.

In some embodiments, administration of about 6×10⁹ or more NK cells (e.g., expanded NK cells) to a subject having Parkinson's disease improves the subject's left leg movement by about 30% as compared to the subject's left leg movement prior to administration of the NK cells.

In some embodiments, administration of about 8×10⁹ or more NK cells (e.g., expanded NK cells) to a subject having Parkinson's disease improves the subject's left leg movement by about 30% as compared to the subject's left leg movement prior to administration of the NK cells.

In some embodiments, administration of about 4×10⁹ or more NK cells (e.g., expanded NK cells) to a subject having Parkinson's disease improves the subject's left two finger stickiness by about 30% as compared to the subject's left two finger stickiness prior to administration of the NK cells.

In some embodiments, administration of about 6×10⁹ or more NK cells (e.g., expanded NK cells) to a subject having Parkinson's disease improves the subject's left two finger stickiness by about 30% as compared to the subject's left two finger stickiness prior to administration of the NK cells.

In some embodiments, administration of about 8×10⁹ or more NK cells (e.g., expanded NK cells) to a subject having Parkinson's disease improves the subject's left two finger stickiness by about 30% as compared to the subject's left two finger stickiness prior to administration of the NK cells.

In some embodiments, administration of about 4×10⁹ or more NK cells (e.g., expanded NK cells) to a subject having Parkinson's disease improves the subject's face stiffness/smiling by about 30% as compared to the subject's face stiffness/smiling prior to administration of the NK cells.

In some embodiments, administration of about 6×10⁹ or more NK cells (e.g., expanded NK cells) to a subject having Parkinson's disease improves the subject's face stiffness/smiling by about 30% as compared to the subject's face stiffness/smiling prior to administration of the NK cells.

In some embodiments, administration of about 8×10⁹ or more NK cells (e.g., expanded NK cells) to a subject having Parkinson's disease improves the subject's face stiffness/smiling by about 30% as compared to the subject's face stiffness/smiling prior to administration of the NK cells.

In some embodiments, administration of about 4×10⁹ or more NK cells (e.g., expanded NK cells) to a subject having Parkinson's disease improves the subject's depression by about 30% as compared to the subject's depression prior to administration of the NK cells.

In some embodiments, administration of about 6×10⁹ or more NK cells (e.g., expanded NK cells) to a subject having Parkinson's disease improves the subject's depression by about 30% as compared to the subject's depression prior to administration of the NK cells.

In some embodiments, administration of about 4×10⁹ or more NK cells (e.g., expanded NK cells) to a subject having Parkinson's disease improves the subject's depression by about 30% as compared to the subject's depression prior to administration of the NK cells.

In some embodiments, administration of about 6×10⁹ or more NK cells (e.g., expanded NK cells) to a subject having Parkinson's disease improves the subject's depression by about 30% as compared to the subject's depression prior to administration of the NK cells.

In some embodiments, administration of about 8×10⁹ or more NK cells (e.g., expanded NK cells) to a subject having Parkinson's disease improves the subject's depression by about 30% as compared to the subject's depression prior to administration of the NK cells.

In some embodiments, administration of about 4×10⁹ or more NK cells (e.g., expanded NK cells) to a subject having Parkinson's disease improves the subject's neck stiffness by about 25% as compared to the subject's neck stiffness prior to administration of the NK cells.

In some embodiments, administration of about 6×10⁹ or more NK cells (e.g., expanded NK cells) to a subject having Parkinson's disease improves the subject's neck stiffness by about 25% as compared to the subject's neck stiffness prior to administration of the NK cells.

In some embodiments, administration of about 8×10⁹ or more NK cells (e.g., expanded NK cells) to a subject having Parkinson's disease improves the subject's neck stiffness by about 25% as compared to the subject's neck stiffness prior to administration of the NK cells.

In some embodiments, administration of about 4×10⁹ or more NK cells (e.g., expanded NK cells) to a subject having Parkinson's disease improves the subject's left side weakness by about 25% as compared to the subject's mental left side weakness prior to administration of the NK cells.

In some embodiments, administration of about 6×10⁹ or more NK cells (e.g., expanded NK cells) to a subject having Parkinson's disease improves the subject's left side weakness by about 25% as compared to the subject's mental left side weakness prior to administration of the NK cells.

In some embodiments, administration of about 8×10⁹ or more NK cells (e.g., expanded NK cells) to a subject having Parkinson's disease improves the subject's left side weakness by about 25% as compared to the subject's mental left side weakness prior to administration of the NK cells.

In some embodiments, administration of about 4×10⁹ or more NK cells (e.g., expanded NK cells) to a subject having Parkinson's disease improves the subject's left hand movement by about 20% as compared to the subject's left hand movement prior to administration of the NK cells.

In some embodiments, administration of about 6×10⁹ or more NK cells (e.g., expanded NK cells) to a subject having Parkinson's disease improves the subject's left hand movement by about 20% as compared to the subject's left hand movement prior to administration of the NK cells.

In some embodiments, administration of about 8×10⁹ or more NK cells (e.g., expanded NK cells) to a subject having Parkinson's disease improves the subject's left hand movement by about 20% as compared to the subject's left hand movement prior to administration of the NK cells.

In some embodiments, administration of about 4×10⁹ or more NK cells (e.g., expanded NK cells) to a subject having Parkinson's disease improves the subject's balance by about 20% as compared to the subject's balance prior to administration of the NK cells.

In some embodiments, administration of about 6×10⁹ or more NK cells (e.g., expanded NK cells) to a subject having Parkinson's disease improves the subject's balance by about 20% as compared to the subject's balance prior to administration of the NK cells.

In some embodiments, administration of about 8×10⁹ or more NK cells (e.g., expanded NK cells) to a subject having Parkinson's disease improves the subject's balance by about 20% as compared to the subject's balance prior to administration of the NK cells.

In some embodiments, administration of the NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 6×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 8×10⁹ or more NK cells (e.g., expanded NK cells) to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

In some embodiments, administration of about 4×10⁹ or more NK cells (e.g., expanded NK cells) to a subject having Parkinson's disease improves the subject's mental sharpness/reaction by about 40% within about 48 hours of NK cell administration as compared to the subject's mental sharpness/reaction prior to administration of the NK cells. In some embodiments, administration of about 6×10⁹ or more NK cells to a subject having Parkinson's disease improves the subject's mental sharpness/reaction by about 40% within about 48 hours of NK cell administration as compared to the subject's mental sharpness/reaction prior to administration of the NK cells. In some embodiments, administration of about 8×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

In some embodiments, administration of about 4×10⁹ or more NK cells (e.g., expanded NK cells) to a subject having Parkinson's disease improves the subject's fatigue/energy by about 40% within about 48 hours of NK cell administration as compared to the subject's fatigue/energy prior to administration of the NK cells. In some embodiments, administration of about 6×10⁹ or more NK cells to a subject having Parkinson's disease improves the subject's fatigue/energy by about 40% within about 48 hours of NK cell administration as compared to the subject's fatigue/energy prior to administration of the NK cells. In some embodiments, administration of about 8×10⁹ or more NK cells to a subject having Parkinson's disease improves the subject's fatigue/energy by about 40% within about 48 hours of NK cell administration as compared to the subject's fatigue/energy prior to administration of the NK cells.

In some embodiments, administration of about 4×10⁹ or more NK cells (e.g., expanded NK cells) to a subject having Parkinson's disease improves the subject's speech by about 40% within about 48 hours of NK cell administration as compared to the subject's speech prior to administration of the NK cells.

In some embodiments, administration of about 6×10⁹ or more NK cells (e.g., expanded NK cells) to a subject having Parkinson's disease improves the subject's speech by about 40% within about 48 hours of NK cell administration as compared to the subject's speech prior to administration of the NK cells.

In some embodiments, administration of about 8×10⁹ or more NK cells (e.g., expanded NK cells) to a subject having Parkinson's disease improves the subject's speech by about 40% within about 48 hours of NK cell administration as compared to the subject's speech prior to administration of the NK cells.

In some embodiments, administration of about 4×10⁹ or more NK cells (e.g., expanded NK cells) to a subject having Parkinson's disease improves the subject's left leg movement by about 30% within about 48 hours of NK cell administration as compared to the subject's left leg movement prior to administration of the NK cells. In some embodiments, administration of about 6×10⁹ or more NK cells to a subject having Parkinson's disease improves the subject's left leg movement by about 30% within about 48 hours of NK cell administration as compared to the subject's left leg movement prior to administration of the NK cells. In some embodiments, administration of about 8×10⁹ or more NK cells to a subject having Parkinson's disease improves the subject's left leg movement by about 30% within about 48 hours of NK cell administration as compared to the subject's left leg movement prior to administration of the NK cells.

In some embodiments, administration of about 4×10⁹ or more NK cells (e.g., expanded NK cells) to a subject having Parkinson's disease improves the subject's left two finger stickiness by about 30% within about 48 hours of NK cell administration as compared to the subject's left two finger stickiness prior to administration of the NK cells. In some embodiments, administration of about 6×10⁹ or more NK cells to a subject having Parkinson's disease improves the subject's left two finger stickiness by about 30% within about 48 hours of NK cell administration as compared to the subject's left two finger stickiness prior to administration of the NK cells. In some embodiments, administration of about 8×10⁹ or more NK cells to a subject having Parkinson's disease improves the subject's left two finger stickiness by about 30% within about 48 hours of NK cell administration as compared to the subject's left two finger stickiness prior to administration of the NK cells.

In some embodiments, administration of about 4×10⁹ or more NK cells (e.g., expanded NK cells) to a subject having Parkinson's disease improves the subject's face stiffness/smiling by about 30% within about 48 hours of NK cell administration as compared to the subject's face stiffness/smiling prior to administration of the NK cells. In some embodiments, administration of about 6×10⁹ or more NK cells to a subject having Parkinson's disease improves the subject's face stiffness/smiling by about 30% within about 48 hours of NK cell administration as compared to the subject's face stiffness/smiling prior to administration of the NK cells. In some embodiments, administration of about 8×10⁹ or more NK cells to a subject having Parkinson's disease improves the subject's face stiffness/smiling by about 30% within about 48 hours of NK cell administration as compared to the subject's face stiffness/smiling prior to administration of the NK cells.

In some embodiments, administration of about 4×10⁹ or more NK cells (e.g., expanded NK cells) to a subject having Parkinson's disease improves the subject's depression by about 30% within about 48 hours of NK cell administration as compared to the subject's depression prior to administration of the NK cells. In some embodiments, administration of about 6×10⁹ or more NK cells to a subject having Parkinson's disease improves the subject's depression by about 30% within about 48 hours of NK cell administration as compared to the subject's depression prior to administration of the NK cells. In some embodiments, administration of about 8×10⁹ or more NK cells to a subject having Parkinson's disease improves the subject's depression by about 30% within about 48 hours of NK cell administration as compared to the subject's depression prior to administration of the NK cells.

In some embodiments, administration of about 4×10⁹ or more NK cells (e.g., expanded NK cells) to a subject having Parkinson's disease improves the subject's depression by about 30% within about 48 hours of NK cell administration as compared to the subject's depression prior to administration of the NK cells. In some embodiments, administration of about 6×10⁹ or more NK cells to a subject having Parkinson's disease improves the subject's depression by about 30% within about 48 hours of NK cell administration as compared to the subject's depression prior to administration of the NK cells. In some embodiments, administration of about 8×10⁹ or more NK cells to a subject having Parkinson's disease improves the subject's depression by about 30% within about 48 hours of NK cell administration as compared to the subject's depression prior to administration of the NK cells.

In some embodiments, administration of about 4×10⁹ or more NK cells (e.g., expanded NK cells) to a subject having Parkinson's disease improves the subject's neck stiffness by about 25% within about 48 hours of NK cell administration as compared to the subject's neck stiffness prior to administration of the NK cells. In some embodiments, administration of about 6×10⁹ or more NK cells to a subject having Parkinson's disease improves the subject's neck stiffness by about 25% within about 48 hours of NK cell administration as compared to the subject's neck stiffness prior to administration of the NK cells. In some embodiments, administration of about 8×10⁹ or more NK cells to a subject having Parkinson's disease improves the subject's neck stiffness by about 25% within about 48 hours of NK cell administration as compared to the subject's neck stiffness prior to administration of the NK cells.

In some embodiments, administration of about 4×10⁹ or more NK cells (e.g., expanded NK cells) to a subject having Parkinson's disease improves the subject's left side weakness by about 25% within about 48 hours of NK cell administration as compared to the subject's mental left side weakness prior to administration of the NK cells. In some embodiments, administration of about 6×10⁹ or more NK cells to a subject having Parkinson's disease improves the subject's left side weakness by about 25% within about 48 hours of NK cell administration as compared to the subject's mental left side weakness prior to administration of the NK cells. In some embodiments, administration of about 8×10⁹ or more NK cells to a subject having Parkinson's disease improves the subject's left side weakness by about 25% within about 48 hours of NK cell administration as compared to the subject's mental left side weakness prior to administration of the NK cells.

In some embodiments, administration of about 4×10⁹ or more NK cells (e.g., expanded NK cells) to a subject having Parkinson's disease improves the subject's left hand movement by about 20% within about 48 hours of NK cell administration as compared to the subject's left hand movement prior to administration of the NK cells. In some embodiments, administration of about 6×10⁹ or more NK cells to a subject having Parkinson's disease improves the subject's left hand movement by about 20% within about 48 hours of NK cell administration as compared to the subject's left hand movement prior to administration of the NK cells. In some embodiments, administration of about 8×10⁹ or more NK cells to a subject having Parkinson's disease improves the subject's left hand movement by about 20% within about 48 hours of NK cell administration as compared to the subject's left hand movement prior to administration of the NK cells.

In some embodiments, administration of about 4×10⁹ or more NK cells (e.g., expanded NK cells) to a subject having Parkinson's disease improves the subject's balance by about 20% within about 48 hours of NK cell administration as compared to the subject's balance prior to administration of the NK cells. In some embodiments, administration of about 6×10⁹ or more NK cells to a subject having Parkinson's disease improves the subject's balance by about 20% within about 48 hours of NK cell administration as compared to the subject's balance prior to administration of the NK cells. In some embodiments, administration of about 8×10⁹ or more NK cells to a subject having Parkinson's disease improves the subject's balance by about 20% within about 48 hours of NK cell administration as compared to the subject's balance prior to administration of the NK cells.

Assessing the therapeutic effects of the treatment on the PD subject can

be done using any suitable option. In some embodiments, the subject provides self-assessments of cognitive impairment, fatigue, speech, facial expression, rigidity in the neck, finger tapping in the left hand, hand movements of the left hand, agility of the left leg, and posture stability in journal entries. In some embodiments subjects, provide self-assessments in terms of perceived percentage improvement.

In some embodiments, cognitive function and non-motor conditions and symptoms are assessed based around the PD rating scales NMSQ (Non-Motor Symptoms Questionnaire). In some embodiments, the physician or evaluator administers the UPDRS or the Movement Disorder Society-Sponsored Revision of the MDS (MSD-UPDRS) to evaluate function and motor symptoms.

In some embodiments, imaging is used to assess improvements. In some embodiments, DaTscan imaging, which uses SPECT (Single Photon Emission Computed Tomography), is used to show how much DAT activity is happening around the subject's striatum, a part of the brain affected by Parkinson's.

In some embodiments, the method further comprises detecting and/or quantifying one or more biomarkers of PD and/or neuroinflammation. In some embodiments, biomarkers for PD include, without limitation, α-synuclein. In some embodiments, the biomarker is a CSF or plasma biomarker. In some embodiments, biomarkers for neuroinflammation is collected via blood or CSF (e.g., before and/or after administering NK cells to the PD subject). In some embodiments, biomarkers for neuroinflammation includes, without limitation, glial fibrillary acidic protein (GFAP), YKL-40, IL-12/IL-23p40, IL-6, IL-8, TNF-α, IL-10, GM-CSF, IL-1β, INF-γ, and/or any combination thereof. In some embodiments, time to L-dopa is used to evaluate the potential worsening of PD which may require the initiation of L-dopa treatment.

In some embodiments, administration of the expanded NK cell population results in decreased neuroinflammation. Also provided is a method of reducing neuroinflammation (e.g., inflammation in the brain related to Parkinson's Disease) by administering a therapeutically effective amount of the expanded NK cells of the present disclosure to a subject in need thereof. In some embodiments, the subject has Parkinson's Disease. In some embodiments, decreased or reduced neuroinflammation is measured based on a decrease in one or more biomarkers of neuroinflammation, as described herein. In some embodiments, the subject's level of neuroinflammation decreases following one or more administrations of the NK cells (e.g., the expanded NK cells). In some embodiments, the subject's level of neuroinflammation decreases by about 1, 2, 3, 4, 5, 6, 7, 8, 9, 10, 20, 25, 30, 40, 50, 60, 70, 75, 80, 90, or 100%, or by an amount in a range that is defined by any two of the preceding values, following one or more administrations of the NK cells. For example, in some embodiments, the subject's level of neuroinflammation decreases by between about 1-100, 1-75, 1-50, 1-25, 1-10, 10-100, 10-75, 10-50, 10-25, 25-100, 25-75, 25-50, 50-100, 50-75, or 75-100%, following one or more administrations of the NK cells. The level of neuroinflammation can be measured using any suitable option. In some embodiments, the level of neuroinflammation is measured by assaying the level or change in level of one or more markers of inflammation in plasma or CSF, e.g., as described herein.

Additional, non-limiting embodiments of the present disclosure are provided in the following numbered arrangements.

1. A method of treating Parkinson's disease (PD) in a subject, the method comprising:
a. identifying a subject, wherein the subject has PD; and
b. administering to the subject an expanded natural killer (NK) cell population, wherein the NK cells are expanded by a method comprising:
   i) isolating at least one of CD56+ cells and/or CD3−/CD56+ cells from the PBMCs;
   ii) co-culturing the at least one of CD56+ cells and/or CD3−/CD56+ cells with a combination of feeder cells in the presence of at least two cytokines;
   iii) wherein the combination of feeder cells comprises irradiated Jurkat cells and irradiated Epstein-Barr virus transformed lymphocyte continuous line (EBV-LCL) cells; and
   iv) wherein the at least two cytokines comprise IL-2 and IL-21.

2. The method of Arrangement 1, wherein the amount of expanded NK cells administered to a subject is a therapeutically effective amount.

3. The method of Arrangement 2, wherein the therapeutically effective amount of expanded NK cells comprises 2×10⁹ to 9×10⁹ cells.

4. The method of Arrangement 1, wherein IL-2 is added at a concentration of 50-1000 IU/mL during step ii).

5. The method of Arrangement 1, wherein IL-21 is added at a concentration of 10-100 ng/ml during step ii).

6. The method of Arrangement 1, further comprising:

co-culturing the at least one of CD56+ cells and/or CD3−/CD56+ cells with the combination of feeder cells, in the presence of IL-2 for a first period; and
co-culturing the at least one of CD56+ cells and/or CD3−/CD56+ cells with the combination of feeder cells, in the presence of IL-21 for a second period.

7. The method of Arrangement 6, wherein IL-21 is added more than once during Day 0-6 of the second period.

8. The method of Arrangement 6, wherein IL-21 and the combination of feeder cells are added more than once during Day 0-6 of the second period.

9. The method of Arrangement 6, wherein IL-21 is added more than once during the first six days of every fourteen-day cycle during the second period.

10. A method of cell therapy comprising:
a. identifying a subject, wherein the subject has PD; and
b. administering to the subject an expanded NK cell population, wherein the NK cells are expanded by a method comprising:
   i) isolating at least one of CD56+ cells and/or CD3−/CD56+ cells from the PBMCs;
   ii) co-culturing the at least one of CD56+ cells and/or CD3−/CD56+ cells with a combination of feeder cells in the presence of at least two cytokines;
   iii) wherein the combination of feeder cells comprises irradiated Jurkat cells and irradiated Epstein-Barr virus transformed lymphocyte continuous line (EBV-LCL) cells; and
   iv) wherein the at least two cytokines comprise IL-2 and IL-21.

11. The method of Arrangement 10, wherein the amount of expanded NK cells administered to a subject is a therapeutically effective amount.

12. The method of Arrangement 11, wherein the therapeutically effective amount of expanded NK cells comprises 2×10⁹ to 9×10⁹ cells.

13. The method of Arrangement 10, wherein IL-2 is added at a concentration of 50-1000 IU/mL during step ii).

14. The method of Arrangement 10, wherein IL-21 is added at a concentration of 10-100 ng/mL during step ii).

15. The method of Arrangement 10, further comprising:
co-culturing the at least one of CD56+ cells and/or CD3−/CD56+ cells with the combination of feeder cells, in the presence of IL-2 for a first period; and
co-culturing the at least one of CD56+ cells and/or CD3−/CD56+ cells with the combination of feeder cells, in the presence of IL-21 for a second period.

16. The method of Arrangement 15, wherein IL-21 is added more than once during Day 0-6 of the second period.

17. The method of Arrangement 15, wherein IL-21 and the combination of feeder cells are added more than once during Day 0-6 of the second period.

18. The method of Arrangement 15, wherein IL-21 is added more than once during the first six days of every fourteen-day cycle during the second period.

19. A population of expanded NK cells, wherein the NK cells were expanded by a method that comprises:
i) isolating at least one of CD56+ cells and/or CD3−/CD56+ cells from the PBMCs;
ii) co-culturing the at least one of CD56+ cells and/or CD3−/CD56+ cells with a combination of feeder cells in the presence of at least two cytokines;
iii) wherein the combination of feeder cells comprises irradiated Jurkat cells and irradiated Epstein-Barr virus transformed lymphocyte continuous line (EBV-LCL) cells; and
iv) wherein the at least two cytokines comprise IL-2 and IL-21; and
wherein the population of expanded NK cells has been administered to a subject who has PD.

20. The population of cells of Arrangement 19, wherein the amount of expanded NK cells administered to a subject is a therapeutically effective amount.

21. The method of Arrangement 20, wherein the therapeutically effective amount of expanded NK cells comprises 2×10⁹ to 9×10⁹ cells.

22. The population of cells of Arrangement 19, wherein IL-2 is added at a concentration of 50-1000 IU/mL during step ii).

23. The population of cells of Arrangement 19, wherein IL-21 is added at a concentration of 10-100 ng/mL during step ii).

24. The population of cells of Arrangement 19, further comprising:
co-culturing the at least one of CD56+ cells and/or CD3−/CD56+ cells with the combination of feeder cells, in the presence of IL-2 for a first period; and
co-culturing the at least one of CD56+ cells and/or CD3−/CD56+ cells with the combination of feeder cells, in the presence of IL-21 for a second period.

25. The population of cells of Arrangement 24, wherein IL-21 is added more than once during Day 0-6 of the second period.

26. The population of cells of Arrangement 24, wherein IL-21 and the combination of feeder cells are added more than once during Day 0-6 of the second period.

27. The population of cells of Arrangement 24, wherein IL-21 is added more than once during the first six days of every fourteen-day cycle during the second period.

28. A method of treating Parkinson's disease (PD) in a subject, the method comprising:
a. identifying a subject, wherein the subject has PD; and
b. administering to the subject a therapeutically effective amount of an autologous NK cell population.

29. The method of Arrangement 28, wherein the NK cells do not include a CAR.

30. The method of Arrangement 28, wherein the NK cells do not include an engineered CAR.

31. The method of any one of the preceding arrangements, wherein about 4×10⁹ NK cells are administered.

32. The method of any one of the preceding arrangements, wherein about 6×10⁹ NK cells are administered.

33. The method of any one of the preceding arrangements, wherein about 8×10⁹ NK cells are administered.

34. The method of any one of the preceding arrangements, wherein administration of the NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

35. The method of any one of the preceding arrangements, wherein administration of the NK cells to a subject having Parkinson's disease improves the subject's mental sharpness/reaction by about 40% as compared to the subject's mental sharpness/reaction prior to administration of the NK cells.

36. The method of any one of the preceding arrangements, wherein administration of the NK cells to a subject having Parkinson's disease improves the subject's fatigue/energy by about 40% as compared to the subject's fatigue/energy prior to administration of the NK cells.

37. The method of any one of the preceding arrangements, wherein administration of the NK cells to a subject having Parkinson's disease improves the subject's speech by about 40% as compared to the subject's speech prior to administration of the NK cells.

38. The method of any one of the preceding arrangements, wherein administration of the NK cells to a subject having Parkinson's disease improves the subject's left leg movement by about 30% as compared to the subject's left leg movement prior to administration of the NK cells.

39. The method of any one of the preceding arrangements, wherein administration of the NK cells to a subject having Parkinson's disease improves the subject's left two finger stickiness by about 30% as compared to the subject's left two finger stickiness prior to administration of the NK cells.

40. The method of any one of the preceding arrangements, wherein administration of the NK cells to a subject having Parkinson's disease improves the subject's face stiffness/smiling by about 30% as compared to the subject's face stiffness/smiling prior to administration of the NK cells.

41. The method of any one of the preceding arrangements, wherein administration of the NK cells to a subject having Parkinson's disease improves the subject's depression by about 30% as compared to the subject's depression prior to administration of the NK cells.

42. The method of any one of the preceding arrangements, wherein administration of the NK cells to a subject having Parkinson's disease improves the subject's neck stiffness by about 25% as compared to the subject's neck stiffness prior to administration of the NK cells.

43. The method of any one of the preceding arrangements, wherein administration of the NK cells to a subject having Parkinson's disease improves the subject's left side weakness by about 25% as compared to the subject's mental left side weakness prior to administration of the NK cells.

44. The method of any one of the preceding arrangements, wherein administration of the NK cells to a subject having Parkinson's disease improves the subject's left hand movement by about 20% as compared to the subject's left hand movement prior to administration of the NK cells.

45. The method of any one of the preceding arrangements, wherein administration of the NK cells to a subject having Parkinson's disease improves the subject's balance by about 20% as compared to the subject's balance prior to administration of the NK cells.

46. The method of any one of the preceding arrangements, wherein administration of about 4×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

47. The method of any one of the preceding arrangements, wherein administration of about 6×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

48. The method of any one of the preceding arrangements, wherein administration of about 8×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

49. The method of any one of the preceding arrangements, wherein administration of about 4×10⁹ or more NK cells to a subject having Parkinson's disease improves the subject's mental sharpness/reaction by about 40% as compared to the subject's mental sharpness/reaction prior to administration of the NK cells.

50. The method of any one of the preceding arrangements, wherein administration of about 6×10⁹ or more NK cells to a subject having Parkinson's disease improves the subject's mental sharpness/reaction by about 40% as compared to the subject's mental sharpness/reaction prior to administration of the NK cells.

51. The method of any one of the preceding arrangements, wherein administration of about 8×10⁹ or more NK cells to a subject having Parkinson's disease improves the subject's mental sharpness/reaction by about 40% as compared to the subject's mental sharpness/reaction prior to administration of the NK cells.

52. The method of any one of the preceding arrangements, wherein administration of about 4×10⁹ or more NK cells to a subject having Parkinson's disease improves the subject's fatigue/energy by about 40% as compared to the subject's fatigue/energy prior to administration of the NK cells.

53. The method of any one of the preceding arrangements, wherein administration of about 6×10⁹ or more NK cells to a subject having Parkinson's disease improves the subject's fatigue/energy by about 40% as compared to the subject's fatigue/energy prior to administration of the NK cells.

54. The method of any one of the preceding arrangements, wherein administration of about 8×10⁹ or more NK cells to a subject having Parkinson's disease improves the subject's fatigue/energy by about 40% as compared to the subject's fatigue/energy prior to administration of the NK cells.

55. The method of any one of the preceding arrangements, wherein administration of about 4×10⁹ or more NK cells to a subject having Parkinson's disease improves the subject's speech by about 40% as compared to the subject's speech prior to administration of the NK cells.

56. The method of any one of the preceding arrangements, wherein administration of about 6×10⁹ or more NK cells to a subject having Parkinson's disease improves the subject's speech by about 40% as compared to the subject's speech prior to administration of the NK cells.

57. The method of any one of the preceding arrangements, wherein administration of about 8×10⁹ or more NK cells to a subject having Parkinson's disease improves the subject's speech by about 40% as compared to the subject's speech prior to administration of the NK cells.

58. The method of any one of the preceding arrangements, wherein administration of about 4×10⁹ or more NK cells to a subject having Parkinson's disease improves the subject's left leg movement by about 30% as compared to the subject's left leg movement prior to administration of the NK cells.

59. The method of any one of the preceding arrangements, wherein administration of about 6×10⁹ or more NK cells to a subject having Parkinson's disease improves the subject's left leg movement by about 30% as compared to the subject's left leg movement prior to administration of the NK cells.

60. The method of any one of the preceding arrangements, wherein administration of about 8×10⁹ or more NK cells to a subject having Parkinson's disease improves the subject's left leg movement by about 30% as compared to the subject's left leg movement prior to administration of the NK cells.

61. The method of any one of the preceding arrangements, wherein administration of about 4×10⁹ or more NK cells to a subject having Parkinson's disease improves the subject's left two finger stickiness by about 30% as compared to the subject's left two finger stickiness prior to administration of the NK cells.

62. The method of any one of the preceding arrangements, wherein administration of about 6×10⁹ or more NK cells to a subject having Parkinson's disease improves the subject's left two finger stickiness by about 30% as compared to the subject's left two finger stickiness prior to administration of the NK cells.

63. The method of any one of the preceding arrangements, wherein administration of about 8×10⁹ or more NK cells to a subject having Parkinson's disease improves the subject's left two finger stickiness by about 30% as compared to the subject's left two finger stickiness prior to administration of the NK cells.

64. The method of any one of the preceding arrangements, wherein administration of about 4×10⁹ or more NK cells to a subject having Parkinson's disease improves the subject's face stiffness/smiling by about 30% as compared to the subject's face stiffness/smiling prior to administration of the NK cells.

65. The method of any one of the preceding arrangements, wherein administration of about 6×10⁹ or more NK cells to a subject having Parkinson's disease improves the subject's face stiffness/smiling by about 30% as compared to the subject's face stiffness/smiling prior to administration of the NK cells.

66. The method of any one of the preceding arrangements, wherein administration of about 8×10⁹ or more NK cells to a subject having Parkinson's disease improves the subject's face stiffness/smiling by about 30% as compared to the subject's face stiffness/smiling prior to administration of the NK cells.

67. The method of any one of the preceding arrangements, wherein administration of about 4×10⁹ or more NK cells to a subject having Parkinson's disease improves the subject's depression by about 30% as compared to the subject's depression prior to administration of the NK cells.

68. The method of any one of the preceding arrangements, wherein administration of about 6×10⁹ or more NK cells to a subject having Parkinson's disease improves the subject's depression by about 30% as compared to the subject's depression prior to administration of the NK cells.

69. The method of any one of the preceding arrangements, wherein administration of about 4×10⁹ or more NK cells to a subject having Parkinson's disease improves the subject's depression by about 30% as compared to the subject's depression prior to administration of the NK cells.

70. The method of any one of the preceding arrangements, wherein administration of about 6×10⁹ or more NK cells to a subject having Parkinson's disease improves the subject's depression by about 30% as compared to the subject's depression prior to administration of the NK cells.

71. The method of any one of the preceding arrangements, wherein administration of about 8×10⁹ or more NK cells to a subject having Parkinson's disease improves the subject's depression by about 30% as compared to the subject's depression prior to administration of the NK cells.

72. The method of any one of the preceding arrangements, wherein administration of about 4×10⁹ or more NK cells to a subject having Parkinson's disease improves the subject's neck stiffness by about 25% as compared to the subject's neck stiffness prior to administration of the NK cells.

73. The method of any one of the preceding arrangements, wherein administration of about 6×10⁹ or more NK cells to a subject having Parkinson's disease improves the subject's neck stiffness by about 25% as compared to the subject's neck stiffness prior to administration of the NK cells.

74. The method of any one of the preceding arrangements, wherein administration of about 8×10⁹ or more NK cells to a subject having Parkinson's disease improves the subject's neck stiffness by about 25% as compared to the subject's neck stiffness prior to administration of the NK cells.

75. The method of any one of the preceding arrangements, wherein administration of about 4×10⁹ or more NK cells to a subject having Parkinson's disease improves the subject's left side weakness by about 25% as compared to the subject's mental left side weakness prior to administration of the NK cells.

76. The method of any one of the preceding arrangements, wherein administration of about 6×10⁹ or more NK cells to a subject having Parkinson's disease improves the subject's left side weakness by about 25% as compared to the subject's mental left side weakness prior to administration of the NK cells.

77. The method of any one of the preceding arrangements, wherein administration of about 8×10⁹ or more NK cells to a subject having Parkinson's disease improves the subject's left side weakness by about 25% as compared to the subject's mental left side weakness prior to administration of the NK cells.

78. The method of any one of the preceding arrangements, wherein administration of about 4×10⁹ or more NK cells to a subject having Parkinson's disease improves the subject's left hand movement by about 20% as compared to the subject's left hand movement prior to administration of the NK cells.

79. The method of any one of the preceding arrangements, wherein administration of about 6×10⁹ or more NK cells to a subject having Parkinson's disease improves the subject's left hand movement by about 20% as compared to the subject's left hand movement prior to administration of the NK cells.

80. The method of any one of the preceding arrangements, wherein administration of about 8×10⁹ or more NK cells to a subject having Parkinson's disease improves the subject's left hand movement by about 20% as compared to the subject's left hand movement prior to administration of the NK cells.

81. The method of any one of the preceding arrangements, wherein administration of about 4×10⁹ or more NK cells to a subject having Parkinson's disease improves the subject's balance by about 20% as compared to the subject's balance prior to administration of the NK cells.

82. The method of any one of the preceding arrangements, wherein administration of about 6×10⁹ or more NK cells to a subject having Parkinson's disease improves the subject's balance by about 20% as compared to the subject's balance prior to administration of the NK cells.

83. The method of any one of the preceding arrangements, wherein administration of about 8×10⁹ or more NK cells to a subject having Parkinson's disease improves the subject's balance by about 20% as compared to the subject's balance prior to administration of the NK cells.

84. The method of any one of the preceding arrangements, wherein administration of the NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

85. The method of any one of the preceding arrangements, wherein administration of about 4×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

86. The method of any one of the preceding arrangements, wherein administration of about 6×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

87. The method of any one of the preceding arrangements, wherein administration of about 8×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% within about 48 hours of NK cell administration as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

88. The method of any one of the preceding arrangements, wherein administration of about 4×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

89. The method of any one of the preceding arrangements, wherein administration of about 6×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

90. The method of any one of the preceding arrangements, wherein administration of about 8×10⁹ or more NK cells to a subject having Parkinson's disease improves the subject's mental sharpness/reaction by about 40% within about 48 hours of NK cell administration as compared to the subject's mental sharpness/reaction prior to administration of the NK cells.

91. The method of any one of the preceding arrangements, wherein administration of about 4×10⁹ or more NK cells to a subject having Parkinson's disease improves the subject's fatigue/energy by about 40% within about 48 hours of NK cell administration as compared to the subject's fatigue/energy prior to administration of the NK cells.

92. The method of any one of the preceding arrangements, wherein administration of about 6×10⁹ or more NK cells to a subject having Parkinson's disease improves the subject's fatigue/energy by about 40% within about 48 hours of NK cell administration as compared to the subject's fatigue/energy prior to administration of the NK cells.

93. The method of any one of the preceding arrangements, wherein administration of about 8×10⁹ or more NK cells to a subject having Parkinson's disease improves the subject's fatigue/energy by about 40% within about 48 hours of NK cell administration as compared to the subject's fatigue/energy prior to administration of the NK cells.

94. The method of any one of the preceding arrangements, wherein administration of about 4×10⁹ or more NK cells to a subject having Parkinson's disease improves the subject's speech by about 40% within about 48 hours of NK cell administration as compared to the subject's speech prior to administration of the NK cells.

95. The method of any one of the preceding arrangements, wherein administration of about 6×10⁹ or more NK cells to a subject having Parkinson's disease improves the subject's speech by about 40% within about 48 hours of NK cell administration as compared to the subject's speech prior to administration of the NK cells.

96. The method of any one of the preceding arrangements, wherein administration of about 8×10⁹ or more NK cells to a subject having Parkinson's disease improves the subject's speech by about 40% within about 48 hours of NK cell administration as compared to the subject's speech prior to administration of the NK cells.

97. The method of any one of the preceding arrangements, wherein administration of about 4×10⁹ or more NK cells to a subject having Parkinson's disease improves the subject's left leg movement by about 30% within about 48 hours of NK cell administration as compared to the subject's left leg movement prior to administration of the NK cells.

98. The method of any one of the preceding arrangements, wherein administration of about 6×10⁹ or more NK cells to a subject having Parkinson's disease improves the subject's left leg movement by about 30% within about 48 hours of NK cell administration as compared to the subject's left leg movement prior to administration of the NK cells.

99. The method of any one of the preceding arrangements, wherein administration of about 8×10⁹ or more NK cells to a subject having Parkinson's disease improves the subject's left leg movement by about 30% within about 48 hours of NK cell administration as compared to the subject's left leg movement prior to administration of the NK cells.

100. The method of any one of the preceding arrangements, wherein administration of about 4×10⁹ or more NK cells to a subject having Parkinson's disease improves the subject's left two finger stickiness by about 30% within about 48 hours of NK cell administration as compared to the subject's left two finger stickiness prior to administration of the NK cells.

101. The method of any one of the preceding arrangements, wherein administration of about 6×10⁹ or more NK cells to a subject having Parkinson's disease improves the subject's left two finger stickiness by about 30% within about 48 hours of NK cell administration as compared to the subject's left two finger stickiness prior to administration of the NK cells.

102. The method of any one of the preceding arrangements, wherein administration of about 8×10⁹ or more NK cells to a subject having Parkinson's disease improves the subject's left two finger stickiness by about 30% within about 48 hours of NK cell administration as compared to the subject's left two finger stickiness prior to administration of the NK cells.

103. The method of any one of the preceding arrangements, wherein administration of about 4×10⁹ or more NK cells to a subject having Parkinson's disease improves the subject's face stiffness/smiling by about 30% within about 48 hours of NK cell administration as compared to the subject's face stiffness/smiling prior to administration of the NK cells.

104. The method of any one of the preceding arrangements, wherein administration of about 6×10⁹ or more NK cells to a subject having Parkinson's disease improves the subject's face stiffness/smiling by about 30% within about 48 hours of NK cell administration as compared to the subject's face stiffness/smiling prior to administration of the NK cells.

105. The method of any one of the preceding arrangements, wherein administration of about 8×10⁹ or more NK cells to a subject having Parkinson's disease improves the subject's face stiffness/smiling by about 30% within about 48 hours of NK cell administration as compared to the subject's face stiffness/smiling prior to administration of the NK cells.

106. The method of any one of the preceding arrangements, wherein administration of about 4×10⁹ or more NK cells to a subject having Parkinson's disease improves the subject's depression by about 30% within about 48 hours of NK cell administration as compared to the subject's depression prior to administration of the NK cells.

107. The method of any one of the preceding arrangements, wherein administration of about 6×10⁹ or more NK cells to a subject having Parkinson's disease improves the subject's depression by about 30% within about 48 hours of NK cell administration as compared to the subject's depression prior to administration of the NK cells.

108. The method of any one of the preceding arrangements, wherein administration of about 4×10⁹ or more NK cells to a subject having Parkinson's disease improves the subject's depression by about 30% within about 48 hours of NK cell administration as compared to the subject's depression prior to administration of the NK cells.

109. The method of any one of the preceding arrangements, wherein administration of about 6×10⁹ or more NK cells to a subject having Parkinson's disease improves the subject's depression by about 30% within about 48 hours of NK cell administration as compared to the subject's depression prior to administration of the NK cells.

110. The method of any one of the preceding arrangements, wherein administration of about 8×10⁹ or more NK cells to a subject having Parkinson's disease improves the subject's depression by about 30% within about 48 hours of NK cell administration as compared to the subject's depression prior to administration of the NK cells.

111. The method of any one of the preceding arrangements, wherein administration of about 4×10⁹ or more NK cells to a subject having Parkinson's disease improves the subject's neck stiffness by about 25% within about 48 hours of NK cell administration as compared to the subject's neck stiffness prior to administration of the NK cells.

112. The method of any one of the preceding arrangements, wherein administration of about 6×10⁹ or more NK cells to a subject having Parkinson's disease improves the subject's neck stiffness by about 25% within about 48 hours of NK cell administration as compared to the subject's neck stiffness prior to administration of the NK cells.

113. The method of any one of the preceding arrangements, wherein administration of about 8×10⁹ or more NK cells to a subject having Parkinson's disease improves the subject's neck stiffness by about 25% within about 48 hours of NK cell administration as compared to the subject's neck stiffness prior to administration of the NK cells.

114. The method of any one of the preceding arrangements, wherein administration of about 4×10⁹ or more NK cells to a subject having Parkinson's disease improves the subject's left side weakness by about 25% within about 48 hours of NK cell administration as compared to the subject's mental left side weakness prior to administration of the NK cells.

115. The method of any one of the preceding arrangements, wherein administration of about 6×10⁹ or more NK cells to a subject having Parkinson's disease improves the subject's left side weakness by about 25% within about 48 hours of NK cell administration as compared to the subject's mental left side weakness prior to administration of the NK cells.

116. The method of any one of the preceding arrangements, wherein administration of about 8×10⁹ or more NK cells to a subject having Parkinson's disease improves the subject's left side weakness by about 25% within about 48 hours of NK cell administration as compared to the subject's mental left side weakness prior to administration of the NK cells.

117. The method of any one of the preceding arrangements, wherein administration of about 4×10⁹ or more NK cells to a subject having Parkinson's disease improves the subject's left hand movement by about 20% within about 48 hours of NK cell administration as compared to the subject's left hand movement prior to administration of the NK cells.

118. The method of any one of the preceding arrangements, wherein administration of about 6×10⁹ or more NK cells to a subject having Parkinson's disease improves the subject's left hand movement by about 20% within about 48 hours of NK cell administration as compared to the subject's left hand movement prior to administration of the NK cells.

119. The method of any one of the preceding arrangements, wherein administration of about 8×10⁹ or more NK cells to a subject having Parkinson's disease improves the subject's left hand movement by about 20% within about 48 hours of NK cell administration as compared to the subject's left hand movement prior to administration of the NK cells.

120. The method of any one of the preceding arrangements, wherein administration of about 4×10⁹ or more NK cells to a subject having Parkinson's disease improves the subject's balance by about 20% within about 48 hours of NK cell administration as compared to the subject's balance prior to administration of the NK cells.

121. The method of any one of the preceding arrangements, wherein administration of about 6×10⁹ or more NK cells to a subject having Parkinson's disease improves the subject's balance by about 20% within about 48 hours of NK cell administration as compared to the subject's balance prior to administration of the NK cells.

122. The method of any one of the preceding arrangements, wherein administration of about 8×10⁹ or more NK cells to a subject having Parkinson's disease improves the subject's balance by about 20% within about 48 hours of NK cell administration as compared to the subject's balance prior to administration of the NK cells.

123. The method of any one of the preceding arrangements, wherein administration of the NK cells decreases neuroinflammation in the subject as compared to the level of neuroinflammation in the subject prior to administration of the NK cells.

124. The method of any one of the preceding claims, wherein the expanded NK cell population or the autologous NK cell population is or comprises SNK01.

### EXAMPLES

The following examples are provided to illustrate certain particular features and/or embodiments. These examples should not be construed to limit the disclosure to the particular features or embodiments described.

### Example 1

### Production of CD56+ Natural Killer (NK) Cells

CD56+ cells and CD3−/CD56+ cells are isolated from PBMCs by the following method. First, the PBMCs are isolated from the blood using a Ficoll-Hypaque density gradient method and then the cells are counted.

### Example 1-1

### Preparation for Producing CD56+ Cells

The counted PBMCs are added with a MACS buffer (1×PBS+0.5% HSA) and suspended, and added with CD56 microbeads (Miltenyi Biotec) to be 1 to 20 μL per 1.0×10⁷ PBMCs, and then incubated at 2 to 8° C. for 5 to 30 minutes. After incubation, the MACS buffer is added and mixed, and then the mixture is centrifuged (600×g) to precipitate the cells. After centrifugation, a supernatant is removed, and the cells are suspended by adding the MACS buffer and added in a column connected to a MACS separator. The MACS buffer is passed through the column to remove non-specific binding. The column is separated from the MACS separator and transferred to a 15 mL conical tube, and then added with the MACS buffer to isolate CD56+ cells attached to the column.

### Example 1-2

### Preparation for Producing CD3−/CD56+ Cells

The counted PBMCs are added with a MACS buffer (1×PBS±0.5% HSA) and suspended, and added with CD3 microbeads (Miltenyi Biotec) to be 1 to 20 μL per 1.0×10⁷ PBMCs, and then incubated at 2 to 8° C. for 5 to 30 minutes. After incubation, the MACS buffer is added and mixed, and then the mixture is centrifuged (600×g) to precipitate the cells. After centrifugation, a supernatant is removed, and the cells are suspended by adding the MACS buffer and added in a column connected to a MACS separator. The MACS buffer passed through the column to collect CD3-cells. The collected CD3-cells are added with a MACS buffer (1×PBS+0.5% HSA) and suspended, and added with CD56 microbeads (Miltenyi Biotec) to be 1 to 20 μL per 1.0×10⁷ CD3-cells, and then incubated at 2 to 8° C. for 5 to 30 minutes. After incubation, the MACS buffer is added and mixed, and then the mixture is centrifuged (600×g) to precipitate the cells. After centrifugation, a supernatant is removed, and the cells are suspended by adding the MACS buffer and added in a column connected to a MACS separator. The MACS buffer is passed through the column to remove non-specific binding. The column is separated from the MACS separator and transferred to a 15 mL conical tube, and then added with the MACS buffer to isolate CD3−/CD56+ cells attached to the column.

### Example 1-3

### Production of NK Cells Using the CD56+ Cells and CD3−/CD56+ Cells

The CD56+ cells or the CD3−/CD56+ cells isolated from the PBMCs as in Examples 1-1 and 1-2 are added in a RPMI-1640 medium containing FBS 10% added with IL-2 at a concentration of 500 IU/mL together with prepared combination of feeder cells (Jurkat cells and EBV-LCL cells) irradiated with 100 Gy radiation and then co-cultured in an incubator at 37° C. and 5% CO₂. The ratio of (CD56+ cells and/or CD3−/CD56+ cells):(Jurkat cells):(EBV-LCL cells) is about 1:30:30.

Meanwhile, the Jurkat cells may be obtained from ATCC (ATCC TIB-152), and the EBV-LCL cells are prepared by the following method: 30×10⁶ PBMCs are added in 9 mL of a culture medium, the mixture is added in a T 25 culture flask, and then 9 m of an EBV supernatant is added. 80 μL of cyclosporine A is added and then cultured at 37° C. After 7 days of culture, a half of supernatant is removed, a fresh culture medium is added, and then 40 μL of cyclosporine A is added. The same process as the 7th day is repeated once every 7 days until 28 days of culture. The cell line is usable after 28 days of culture, and from this time, the cell line is cultured in the culture medium without adding cyclosporine A.

### Example 2

### Production of CD56+ Natural Killer (NK) Cells (IL-2/IL-21 Treated)

NK cells are produced using same method of Example 1 (1-1 to 1-3), except for adding IL-2 (500 IU/mL) and IL-21 (50 ng/mL) instead of IL-2 (500 IU/mL).

### Comparative Example 1

### Production of Natural Killer (NK) Cells Without the CD56+ Cells Isolation Step (IL-2 Treated)

PBMCs are isolated from the blood using a Ficoll-Hypaque density gradient method. The PBMCs are added in a RPMI-1640 medium containing FBS 10% added with IL-2 at a concentration of 500 IU/mL together with prepared feeder cells (Jurkat cells and EBV-LCL cells) irradiated with 100 Gy radiation and then co-cultured in an incubator at 37° C. and 5% CO₂.

### Comparative Example 2

### Production of Natural Killer (NK) Cells Without the CD56+ Cells Isolation Step (IL-2/IL-21 Treated)

NK cells are produced using same method of Comparative Example 1, except for adding IL-2 (500 IU/mL) and IL-21 (50 ng/mL) instead of IL-2 (500 IU/mL).

### Comparative Examples 3&4

### Production of Natural Killer (NK) Cells Without the CD56+ Cells Isolation Step

NK cells are produced using similar methods of Comparative Examples 1&2, respectively, except for that a ratio of PBMC:(Jurkat cells):(EBV-LCL cells) is 1:0.5:0.5.

### Experimental Example 5

### Treatment of PD Patients with NK Cells

CD56+ NK cells are produced according to the method of Examples 1, 2 and Comparative Examples 1, 2 for 18 days, except that PBMCs of PD patients are used. With respect to each of the NK cells cultured in a CO₂ incubator according to Examples 1, 2 and Comparative Examples 1, 2, on Day 6 of culture in a T 25 culture flask, cells are inoculated into a 350 mL bag on the basis of the cell number of 1.0×10⁵ to 2.0×10⁶/mL and further cultured for 4 days. On Day 10 of culture, the cells are inoculated into a 1 L bag on the basis of the cell number of 1.0×10⁵ to 2.0×10⁶/mL and then further cultured for 4 days. Finally, on Day 14 of culture, the cells are inoculated into a 1 L bag on the basis of the cell number of 1.0×10⁵ to 2.0×10⁶/mL and then further cultured for 3 to 6 days.

Parkinson's Disease patients are grouped randomly and marked. The control group will not be injected with NK cells. The NK cell-treated group is injected six times with 1-3×10⁷ NK cells/per kg of body weight and 500 IU/mL of IL-2 at weekly intervals intravenously. NK cells are added repeatedly until improvement in PD symptoms is achieved.

Cognitive and motor functions of the patient is monitored at 1, 3, 6, 12 months. After 12 months, the NK cell-treated group will exhibit improved cognitive and motor functions.

### Experimental Example 6

### Determination of which NK Cells Most Efficiently Internalize and Degrade α-Synuclein

Three different types of NK cells: NK-92 cells, primary NK cells, and culture expanded NK cells (SNK) are evaluated to determine which cell type is most efficient at internalizing and degrading α-synuclein. An α-synuclein preparation is prepared and comprise monomers, oligomers, and high molecular weight fibrils. The NK cells are treated with various concentrations of α-synuclein for 1 hour. The level of internalization of α-synuclein is evaluated by western blot. The level of degradation of α-synuclein at the time point of 1, 4, 12, 24 hours after α-synuclein treatment is evaluated by western blot.

### Example 7

### Assessing the Effect of NK Cells in a Subject with Parkinson's Disease

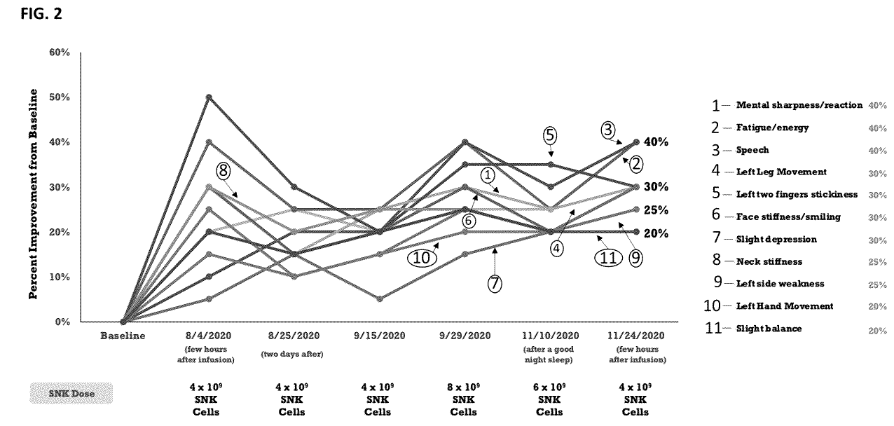

The effect of SNK cells on mental sharpness/reaction, fatigue/energy, speech, left leg movement, left two finger stickiness, face stiffness/smiling, depression, neck stiffness, left side weakness, left hand movement, and balance, were assessed in a subject having PD, who was administered between 4×10⁹ and 8×10⁹ NK cells according to the dosing schedule shown in FIG. 2. A 47-year-old individual with several years history of progressing PD received SIX infusions of SNK01 (www(dot)sec(dot)gov/ix?doc=/Archives/edgar/data/1845459/000110465923074785/gfor-20230331xs4a.htm) over four months in 2020 under compassionate use. Treatment was suspended due to COVID-19 restrictions. The subject reported on cognitive impairment, fatigue, speech, facial expression, rigidity in the neck, finger tapping in the left hand, hand movements of the left hand, agility of the left leg, and posture stability.

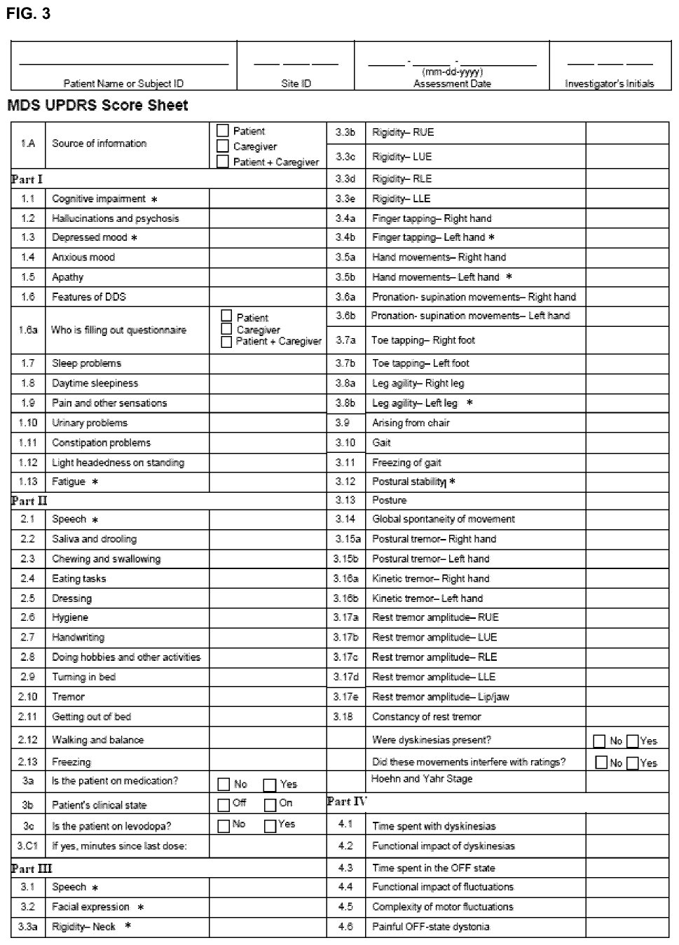

Self-assessments were made by the subject and reported in journal entries and contained components of the MDS-UPDRS (Unified Parkinson's Disease Rating Scale) (FIG. 3), which provided some motor and non-motor aspects of the subject's PD symptoms. Subjects self-reported in terms of perceived percentage improvement. The reports were mostly in Part III of the questionnaire (FIG. 3), which is related to motor outcomes. A couple of items were in Part I, which are the non-motor scores. The topics the subject reported on are marked with * in FIG. 3 (except for left-side weakness).

At the end of the treatment period after six doses, the patient reported they had improved cognition, speech, energy, balance, and overall muscle strength and movement (FIG. 2). The patient did not report any adverse reactions related to SNK01 treatment. SNK01 is an autologous NK cell therapy (www(dot)sec(dot)gov/ix?doc=/Archives/edgar/data/1845459/000110465923074785/gfor-20230331xs4a.htm).

Prior to administration of the NK cells, baseline mental sharpness/reaction, fatigue/energy, speech, left leg movement, left two finger stickiness, face stiffness/smiling, depression, neck stiffness, left side weakness, left hand movement, and balance, were assessed. On Day 1, 4×10⁹ NK cells were intravenously administered to the subject. Within hours of administration, the subject's mental sharpness/reaction, fatigue/energy, speech, left leg movement, left two finger stickiness, face stiffness/smiling, depression, neck stiffness, left side weakness, left hand movement, and balance, were assessed.

As can be seen in FIG. 2, the subject treated with NK cells showed improved mental sharpness/reaction, fatigue/energy, speech, left leg movement, left two finger stickiness, face stiffness/smiling, depression, neck stiffness, left side weakness, left hand movement, and balance, within a few hours of administration of the NK cells.

On Day 19, 4×10⁹ NK cells were intravenously administered to the subject. The subject's mental sharpness/reaction, fatigue/energy, speech, left leg movement, left two finger stickiness, face stiffness/smiling, depression, neck stiffness, left side weakness, left hand movement, and balance, were assessed 2 days following the administration.

As can be seen in FIG. 2, the subject treated with NK cells maintained improved mental sharpness/reaction, fatigue/energy, speech, left leg movement, left two finger stickiness, face stiffness/smiling, depression, neck stiffness, left side weakness, left hand movement, and balance, for at least two days after administration of the NK cells.

On Day 42, 4×10⁹ NK cells were intravenously administered to the subject, and the subject's mental sharpness/reaction, fatigue/energy, speech, left leg movement, left two finger stickiness, face stiffness/smiling, depression, neck stiffness, left side weakness, left hand movement, and balance, were assessed.

On Day 56, 8×10⁹ NK cells were intravenously administered to the subject, and the subject's mental sharpness/reaction, fatigue/energy, speech, left leg movement, left two finger stickiness, face stiffness/smiling, depression, neck stiffness, left side weakness, left hand movement, and balance, were assessed.

As can be seen in FIG. 2, the subject treated with NK cells demonstrate improved mental sharpness/reaction, fatigue/energy, speech, left leg movement, left two finger stickiness, face stiffness/smiling, depression, neck stiffness, left side weakness, left hand movement, and balance, following administration of larger doses of NK cells.

On Day 98, 6×10⁹ NK cells were intravenously administered to the subject. The subject's mental sharpness/reaction, fatigue/energy, speech, left leg movement, left two finger stickiness, face stiffness/smiling, depression, neck stiffness, left side weakness, left hand movement, and balance, were assessed following a good nights sleep.

As can be seen in FIG. 2, the subject treated with NK cells maintain improved mental sharpness/reaction, fatigue/energy, speech, left leg movement, left two finger stickiness, face stiffness/smiling, depression, neck stiffness, left side weakness, left hand movement, and balance, following administration of 6×10⁹ to 8×10⁹ NK cells.

On Day 112, 4×10⁹ NK cells were intravenously administered to the subject. The subject's mental sharpness/reaction, fatigue/energy, speech, left leg movement, left two finger stickiness, face stiffness/smiling, depression, neck stiffness, left side weakness, left hand movement, and balance, were assessed within hours of administration of the NK cells.

As can be seen in FIG. 2, the subject treated with NK cells over 3-4 months are 40% more mentally sharp/faster reacting as compared to baseline. The subject showed 30% more energy as compared to baseline. The subject showed 40% improved speech, 30% improved left leg movement, 30% improved left two finger stickiness, 30% less face stiffness, 30% less depression, 25% less neck stiffness, 25% less left side weakness, 20% improved left hand movement, and 20% improved balance, as compared to baseline.

### Example 8

### Assessing the Effect of NK Cells in a Subject with Parkinson's Disease

The effect of administering NK cells (e.g., SNK01) to a PD subject (e.g., as described in Experimental Example 5 or Example 7) is assessed. The Cognitive function and non-motor conditions and symptoms are assessed based around the PD rating scales NMSQ (Non-Motor Symptoms Questionnaire). The physician or evaluator administers the UPDRS (Unified Parkinson's Disease Rating Scale) or the Movement Disorder Society-Sponsored Revision of the MDS (MSD-UPDRS).

For imaging, a DaTscan using SPECT (Single Photon Emission Computed Tomography) imaging is used to measure DAT activity around the subject's striatum, a part of the brain affected by Parkinson's. Other biomarkers for neuroinflammation are collected via blood or CSF. Time to L-dopa is used to evaluate the potential worsening of PD, which may warrant initiation of L-dopa treatment.

The foregoing description of the exemplary embodiments has been presented only for the purposes of illustration and description and is not intended to be exhaustive or to limit the invention to the precise forms disclosed. Many modifications and variations are possible in light of the above teaching. It is contemplated that various combinations or sub combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention is defined by the appended claims rather than the foregoing description and the exemplary embodiments described therein.

1. A method of treating Parkinson's disease (PD) in a subject, the method comprising:
a. identifying a subject, wherein the subject has PD; and
b. administering to the subject an expanded natural killer (NK) cell population, wherein the NK cells are expanded by a method comprising:
i) isolating at least one of CD56+ cells and/or CD3−/CD56+ cells from the PBMCs;
ii) co-culturing the at least one of CD56+ cells and/or CD3−/CD56+ cells with a combination of feeder cells in the presence of at least two cytokines;
iii) wherein the combination of feeder cells comprises irradiated Jurkat cells and irradiated Epstein-Barr virus transformed lymphocyte continuous line (EBV-LCL) cells; and
iv) wherein the at least two cytokines comprise IL-2 and IL-21.

2. The method of claim 1, wherein the amount of expanded NK cells administered to a subject is a therapeutically effective amount.

3. The method of claim 2, wherein the therapeutically effective amount of expanded NK cells comprises 2×10⁹ to 9×10⁹ cells.

4. The method of claim 1, wherein IL-2 is added at a concentration of 50-1000 IU/mL during step ii).

5. The method of claim 1, wherein IL-21 is added at a concentration of 10-100 ng/mL during step ii).

6. The method of claim 1, further comprising:
co-culturing the at least one of CD56+ cells and/or CD3−/CD56+ cells with the combination of feeder cells, in the presence of IL-2 for a first period; and
co-culturing the at least one of CD56+ cells and/or CD3−/CD56+ cells with the combination of feeder cells, in the presence of IL-21 for a second period.

6. inal) The method of claim 6, wherein IL-21 is added more than once during Day 0-6 of the second period.

8. The method of claim 6, wherein IL-21 and the combination of feeder cells are added more than once during Day 0-6 of the second period.

9. The method of claim 6, wherein IL-21 is added more than once during the first six days of every fourteen-day cycle during the second period.

10. A method of cell therapy comprising:
a. identifying a subject, wherein the subject has PD; and
b. administering to the subject an expanded NK cell population, wherein the NK cells are expanded by a method comprising:
i) isolating at least one of CD56+ cells and/or CD3−/CD56+ cells from the PBMCs;
ii) co-culturing the at least one of CD56+ cells and/or CD3−/CD56+ cells with a combination of feeder cells in the presence of at least two cytokines;
iii) wherein the combination of feeder cells comprises irradiated Jurkat cells and irradiated Epstein-Barr virus transformed lymphocyte continuous line (EBV-LCL) cells; and
iv) wherein the at least two cytokines comprise IL-2 and IL-21.

11. A method of treating Parkinson's disease (PD) in a subject, the method comprising:
a. identifying a subject, wherein the subject has PD; and
b. administering to the subject a therapeutically effective amount of an autologous NK cell population.

12. The method of claim 1, wherein the NK cells do not include a CAR.

13. The method of claim 1, wherein the NK cells do not include an engineered CAR.

14. The method of claim 1, wherein about 4×10⁹ NK cells are administered.

15. The method of claim 1, wherein about 6×10⁹ NK cells are administered.

16. The method of claim 1, wherein about 8×10⁹ NK cells are administered.

17. The method of claim 1, wherein administration of the NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

18. The method of claim 1, wherein administration of the NK cells to a subject having Parkinson's disease improves the subject's mental sharpness/reaction by about 40% as compared to the subject's mental sharpness/reaction prior to administration of the NK cells.

19. The method of claim 1, wherein administration of about 6×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

20. The method of claim 1, wherein administration of about 8×10⁹ or more NK cells to a subject having Parkinson's disease improves the subject's mental sharpness/reaction, fatigue/energy, speech, left leg movement, left two finger stickiness, face stiffness/smiling, depression, neck stiffness, left side weakness, left hand movement, and/or balance, or any combination thereof, by between about 20-40% as compared to the subject's mental sharpness/reaction, fatigue/energy, speech, left leg movement, left two finger stickiness, face stiffness/smiling, depression, neck stiffness, left side weakness, left hand movement, and/or balance, or any combination thereof, prior to administration of the NK cells.

21. The method of claim 1, wherein administration of about 4×10⁹ or more NK cells to a subject having Parkinson's disease improves the subject's left leg movement by about 30% within about 48 hours of NK cell administration as compared to the subject's left leg movement prior to administration of the NK cells.

22. The method of claim 1, wherein administration of about 4×10⁹ or more NK cells to a subject having Parkinson's disease improves the subject's left two finger stickiness by about 30% within about 48 hours of NK cell administration as compared to the subject's left two finger stickiness prior to administration of the NK cells.

23. The method of claim 1, wherein administration of about 4×10⁹ or more NK cells to a subject having Parkinson's disease improves the subject's face stiffness/smiling by about 30% within about 48 hours of NK cell administration as compared to the subject's face stiffness/smiling prior to administration of the NK cells.

24. The method of claim 1, wherein administration of about 6×10⁹ or more NK cells to a subject having Parkinson's disease improves the subject's balance by about 20% within about 48 hours of NK cell administration as compared to the subject's balance prior to administration of the NK cells.

25. The method of claim 1, wherein administration of the NK cells decreases neuroinflammation in the subject as compared to the level of neuroinflammation in the subject prior to administration of the NK cells.

26. The method of claim 1, wherein the expanded NK cell population or the autologous NK cell population is or comprises SNK01.
